(19) United States
(12) Patent Application Publication
(10) Pub. No.: US 2023/0395235 A1
(43) Pub. Date: Dec. 7, 2023

(54) System and Method for Delivering Personalized Cognitive Intervention

(71) Applicant: NEUROGLEE SCIENCE PTE. LTD., Singapore (SG)

(72) Inventors: Aniketsingh Vithalsingh Rajput, Singapore (SG); Michael Donald Patterson, Singapore (SG); Bikram Aditya Gangwar, Singapore (SG); Iurii Rykov, Singapore (SG)

(21) Appl. No.: 18/033,183

(22) PCT Filed: Oct. 22, 2021
(86) PCT No.: PCT/SG2021/050646

§ 371 Date: Apr. 21, 2023

(30) Foreign Application Priority Data
Oct. 23, 2020 (SG) 10202010516R

(51) Int. Cl. G16H 20/70; A61B 5/01; A61B 5/0205; A61B 5/024; A61B 5/0533; A61B 5/11; A61B 5/16; A61B 5/00

(52) U.S. Cl. CPC G16H 20/70; A61B 5/01; A61B 5/02055; A61B 5/02405; A61B 5/02416; A61B 5/0533; A61B 5/11; A61B 5/162; A61B 5/4088

(57) ABSTRACT

A computational personalized cognitive therapeutic system for treating patients with Mild Cognitive Impairment, Alzheimer's Disease, dementia and related conditions is described. The system includes a patient clinical database, a data aggregation layer and data pre-processor module, a digital cognitive therapy delivery module, a cognitive analytics engine, and a personalised cognitive platform configured to personalize a personalised cognitive digital therapy model. The personalised cognitive digital therapy model defines specific digital treatments to be delivered to the patient using the digital cognitive therapy delivery module each with a different mechanism of action. A range of digital cognitive biomarkers are collected along with behavioural and physiological biomarkers from wearable and medical devices which are processed by the cognitive analytics engine and uses AI/ML methods which are configured to estimate metrics and generate alerts. The metrics are used to assess treatment progress and then personalize the personalised cognitive digital therapy model for the patient including adjustment of digital therapies and medication. Alerts may be generated if adverse side effects are observed. This process is iteratively repeated to provide improved treatment over time.

### CROSS REFERENCE TO RELATED APPLICATIONS

This application is the United States national phase of International Patent Application No.: PCT/SG2021/050646 filed Oct. 22, 2021, and claims priority to Singapore Patent Application No. 10202010516R filed Oct. 23, 2020, the disclosures of which are hereby incorporated by reference in their entireties.

### BACKGROUND OF THE INVENTION

### Field of the Invention

The present disclosure relates to personalised patient treatment systems. In a particular form the present disclosure relates to personalised patient treatment systems for patients with mild cognitive impairment (MCI), Alzheimer's and related conditions including dementia.

### Description of Related Art

Treatment of patients with mild cognitive impairment (MCI), Alzheimer's and related conditions including dementia is challenging. These conditions are complex and treatment often comprises a range of pharmacological interventions (medications) and non-pharmacological interventions such as behaviour modification and cognitive therapies. Unfortunately these medications and combination of these medications including medications for other conditions can create a range of side effects which can be serious and unpredictable. Currently there are only six US FDA approved drugs for Alzheimer's Disease and these medications are known to have massive side effects on the patient's physiological parameters and their ability to perform daily living activities. Some of the side effects include nausea, headache, dizziness, brain bleeding, shortness of breath, heart burn, shakiness, tremors, increase in bowel movements, diarrhoea etc. These side effects arise after intake of medications, and directly affect the patient's physiological parameters and also their quality of life, and unfortunately the influence of the side effects on an individual's physiological patterns and behavioural patterns is typically unpredictable. Thus the medications and interventions used to treat a patient need to be closely monitored and titrated to find the optimal doses of medications and therapy for a particular patient that minimises side effects and provides the greatest therapeutic benefit.

There is thus a need to develop systems and methods to assist clinicians to determine improved treatments for patients including combinations of pharmacological and non-pharmacological interventions that best alleviate symptoms and reduce side effects, particularly in the case of complex conditions, such as mild cognitive impairment (MCI), Alzheimer's and related conditions, or to at least provide a useful alternative to existing systems and methods.

### SUMMARY OF THE INVENTION

Embodiments of a personalised cognitive digital therapeutic system which monitors the effect of therapy on individual patient and can provide valuable insights to the caregiver/clinician for better therapeutic decision making will now be described.

According to a first aspect, there is provided a computational personalized cognitive therapeutic system comprising one or more processors and one or more associated memories configured to implement:
a patient clinical database comprising a data acquisition interface configured to receive and store input data for a plurality of patients from a plurality of heterogeneous data sources, the input data for a patient comprising: personal particular and sociodemographic data; patient medical history and background health data; medication data; clinical data; and wearable data comprising behavioural data and physiological data, wherein the input data sources for a patient comprise one or more wearable devices and one or more of an electronic medical record, a digitised caregiver record, a laboratory information management system, a clinical database, and/or a patient on-boarding module;
a data aggregation and pre-processing module configured to pre-process the input data in the patient clinical database and generate a patient profile for each patient;
a digital cognitive therapy delivery module configured to deliver a plurality of therapies according to a personalised cognitive digital therapy model to a patient using one or more computing devices, each therapy having a different mechanism of action (MOA) and comprising a plurality of adjustable parameters, and the digital cognitive therapy delivery module is further configured to collect a plurality of digital cognitive biomarkers for each therapy, a plurality of behavioural and physiological biomarkers from one or more wearable devices and/or the one or more computing devices, and to measure interactions of the patient with the digital cognitive therapy delivery module, wherein the plurality of therapies comprises at least a cognitive stimulation game therapy, a guided learning therapy, a reminiscence therapy and a physical and mental wellness therapy;
a cognitive analytics engine configured to process the patient profile, digital cognitive biomarkers, and the behavioural and physiological biomarkers using an ensemble of population-based and personalised prediction models trained using a plurality of Artificial Intelligence (AI) and Machine Learning (ML) methods, and which is configured to generate a plurality of metrics to characterize a current cognitive state of the patient and estimate the potential future improvement comprising the probability and size of an expected effect, wherein the plurality of metrics are generated on demand or at least once per day; and
a personalised cognitive platform configured to use the plurality of metrics to personalize the personalised cognitive digital therapy model for each patient by adjusting one or more of the plurality of parameters for one or more of the plurality of therapies to maximise the estimated effect level, and to use the metrics to generate one or more alerts if a therapy does not meet an expected threshold effect level or a side effect exceeds a threshold side effect level to enable adjustment of a medication by a clinician,
wherein the system iteratively refines the personalised cognitive digital therapy model for each patient over time by selecting specific therapies from the plurality of therapies and adjusting the associated adjustable parameters, and obtaining an estimate effect of the adjustments, and after delivery of an adjusted treatment by the digital cognitive therapy delivery module, the cognitive analytics engine generates the plurality of metrics to assess actual effects compared to estimated effects in order to further refine the personalised cognitive digital therapy model by the personalised cognitive platform.

In one form, the data aggregation and pre-processing module is configured to perform data cleaning, dimensionality reduction and data transformation to prepare the input data for further analysis and use by the cognitive analytics engine.

In one form:
the plurality of digital cognitive biomarkers for the cognitive stimulation game therapy comprises one or more of a game specific performance, finger tapping and finger movement related biomarkers, reaction time between a stimulus exposure and a response, and a proxy index of a cognitive load;
the plurality of digital cognitive biomarkers for the guided learning therapy comprises one or more of a quiz result, an answer confidence, a speed of information processing of content, or a time spent with content;
the plurality of digital cognitive biomarkers for the reminiscence therapy comprises one or more of a language marker derived from textual analysis or audio analysis, a speech characteristic derived from audio data of the patient; and
the plurality of digital cognitive biomarkers for the physical and mental wellness therapy comprises one or more of an access frequency of content, a time spent with content, an application opening and closing frequency, a task completion within an allotted time, a compliance with an allotted task, a direct feedback from the patient on the likeability and difficulty of the therapy via a questionnaire, and an emotional expression capture indicating a level of enjoyment.

In one form, the plurality of behavioural and physiological biomarkers comprise one or more of an movement biomarker obtained from an accelerometer and/or a gyroscope, an electrodermal activity or skin conductance biomarker, a photoplethysmography or blood volume pulse biomarker, a heart rate biomarker, a heart rate variability biomarker, a skin temperature biomarker, a facial expression biomarker, an eye tracking biomarker, and a neural activity biomarker.

In one form, the plurality of metrics comprise:
a cognitive baseline pointer which is an estimate of a change in the cognitive state of the patient with respect to a baseline generated using the patient profile and an expected behaviour effect on the patient generated by the cognitive analytics engine;
a mechanism of action pointer for each of the plurality of mechanisms of action which estimates an effect level with respect to an expected effect level for the associated therapy generated by the cognitive analytics engine;
an average mechanism of action pointer which estimates an average effect of the plurality of therapies with respect to an estimate effect generated by the cognitive analytics engine; and
a side effects pointer which measures a severity of one or more side effects.

In a further form, the personalised cognitive platform comprises a MOA management module, an educational content management module and a medication/dose management module, wherein the MOA management module uses at least the mechanism of action pointers and the average mechanism of action pointer to adjust one or more of the plurality of parameters for one or more of the plurality of therapies and to adjust the dosage of each of the plurality of therapies to maximise the estimated effect level of a therapy, and wherein the content education module is configured to adjust a digital content provided to a patient based on the patient's interaction behaviour measured by the digital cognitive therapy delivery module, and the medication/dose management module is configured to record clinical data including medication and dosages and to generate suggested changes to medication and dosages using at least the side effects pointer and the cognitive baseline pointer.

In a further form, the MOA management module is configured to adjust the cognitive stimulation game therapy by adjusting one or more game parameters, game dosage and game timing, and is configured to adjust the guided learning therapy by adjusting the learn amount and timing, and learning content, and is configured to adjust the reminiscence therapy by adjusting the timing of content, content topics and stimulus and is configured to adjust the physical and mental wellness therapy by adjusting a modality, an intensity and a duration of physical exercise or mental exercise.

In one form, the digital cognitive therapy delivery module provides a user interface on a computing device comprising:
a reminder and calendar module configured to allow patients to record reminders and to notify the patient of a scheduled therapy, and monitors therapy compliance;
a note module configured to track goals and record electronic information to assist with daily living activities;
a medication schedule module configured to record a medication schedule and track compliance;
an electronic gratitude journal;
a mood tracker configured to estimate and/or record a mood of a patient, and to provide feedback on past mood history and to provide mood data to a clinician and/or the cognitive analytics engine;
a social media module to facilitate communication with family, friends and support groups;
a gamification system which awards points for completion of therapy or tasks, and rewards for achieving specific points goals, and a comparative score based on treatment progress with respect to other patients with similar diagnosis;
a diet tracking module configured to collect consumption data and provide dietary recommendations; and
a therapy module configured to provide the plurality of therapies to the patient.

In one form, the system comprises a cloud computing platform and the digital cognitive therapy delivery module is configured to execute on one or more patient mobile computing devices.

According to a second aspect, there is provided a method for providing a personalized cognitive therapeutic system comprising:
receiving and storing input data for a plurality of patients from a plurality of heterogeneous data sources in a patient clinical database using a data acquisition interface, the input data for a patient comprising: personal particular and sociodemographic data; patient medical history and background health data; medication data; clinical data; and wearable data comprising behavioural data and physiological data, wherein the input data sources for a patient comprise one or more wearable devices and one or more of an electronic medical record, a digitised caregiver record, a laboratory information management system, a clinical database, and/or a patient on-boarding module;
aggregating and pre-processing the input data in the patient clinical database and generating a patient profile for each patient;
delivering a plurality of therapies according to a personalised cognitive digital therapy model to a patient using one or more computing devices executing a digital cognitive therapy delivery module wherein each therapy has a different mechanism of action (MOA) and comprises a plurality of adjustable parameters, and the digital cognitive therapy delivery module is further configured to collect a plurality of digital cognitive biomarkers for each therapy, a plurality of behavioural and physiological biomarkers from one or more wearable devices and/or the one or more computing devices, and to measure interactions of the patient with the digital cognitive therapy delivery module, wherein the plurality of therapies comprises at least a cognitive stimulation game therapy, a guided learning therapy, a reminiscence therapy and a physical and mental wellness therapy;
processing, using a cognitive analytics engine, the patient profile, the plurality of digital cognitive biomarkers, and the plurality of behavioural and physiological biomarkers using an ensemble of population-based and personalised prediction models trained using a plurality of Artificial Intelligence (AI) and Machine Learning (ML) methods, and which is configured to generate a plurality of metrics to characterize a current cognitive state of the patient and estimate the potential future improvement comprising the probability and size of an expected effect, wherein the plurality of metrics are generated on demand or at least once per day; and
personalizing, by a personalised cognitive platform configured to use the plurality of metrics, the personalised cognitive digital therapy model for each patient by adjusting one or more of the plurality of parameters for one or more of the plurality of therapies to maximise the estimated effect level, and to use the metrics to generate one or more alerts if a therapy does not meet an expected threshold effect level or a side effect exceeds a threshold side effect level to enable adjustment of a medication by a clinician,
wherein the system iteratively refines the personalised cognitive digital therapy model for each patient over time by selecting specific therapies from the plurality of therapies and adjusting the associated adjustable parameters, and obtaining an estimate effect of the adjustments, and after delivery of an adjusted treatment by the digital cognitive therapy delivery module, the cognitive analytics engine generates the plurality of metrics to assess actual effects compared to estimated effects in order to further refine the personalised cognitive digital therapy model by the personalised cognitive platform.

In one form, aggregating and pre-processing comprises performing data cleaning, dimensionality reduction and data transformation to prepare the input data for further analysis and use by the cognitive analytics engine.

In one form:
the plurality of digital cognitive biomarkers for the cognitive stimulation game therapy comprises one or more of a game specific performance, finger tapping and finger movement related biomarkers, reaction time between a stimulus exposure and a response and a proxy index of a cognitive load;
the plurality of digital cognitive biomarkers for the guided learning therapy comprises one or more of a quiz result, an answer confidence, a speed of information processing of content, or a time spent with content;
the plurality of digital cognitive biomarkers for the reminiscence therapy comprises one or more of a language marker derived from textual analysis or audio analysis, a speech characteristic derived from audio data of the patient; and
the plurality of digital cognitive biomarkers for the physical and mental wellness therapy comprises one or more of an access frequency of content, a time spent with content, an application opening and closing frequency, a task completion within an allotted time, a compliance with an allotted task, a direct feedback from the patient on the likeability and difficulty of the therapy via a questionnaire, and an emotional expression capture indicating a level of enjoyment.

In one form, the plurality of behavioural and physiological biomarkers comprise one or more of an movement biomarker obtained from an accelerometer and/or a gyroscope, an electrodermal activity or skin conductance biomarker, a photoplethysmography or blood volume pulse biomarker, a heart rate biomarker, a heart rate variability biomarker, a skin temperature biomarker, a facial expression biomarker, an eye tracking biomarker, and a neural activity biomarker.

In one form, the plurality of metrics comprise:
a cognitive baseline pointer which is an estimate of a change in the cognitive state of the patient with respect to a baseline generated using the patient profile and an expected behaviour effect on the patient generated by the cognitive analytics engine;
a mechanism of action pointer for each of the plurality of mechanisms of action which estimates an effect level with respect to an expected effect level for the associated therapy generated by the cognitive analytics engine;
an average mechanism of action pointer which estimates an average effect of the plurality of therapies with respect to an estimate effect generated by the cognitive analytics engine; and a side effects pointer which measures a severity of one or more side effects.

In a further form, the personalised cognitive platform comprises a MOA management module, an educational content management module and a medication/dose management module, wherein the MOA management module uses at least the mechanism of action pointers and the average mechanism of action pointer to adjust one or more of the plurality of parameters for one or more of the plurality of therapies and to adjust the dosage of each of the plurality of therapies to maximise the estimated effect level of a therapy, and wherein the content education module is configured to adjust a digital content provided to a patient based on the patient's interaction behaviour measured by the digital cognitive therapy delivery module, and the medication/dose management module is configured to record clinical data including medication and dosages and to generate suggested changes to medication and dosages using at least the side effects pointer and the cognitive baseline pointer.

In a further form, the MOA management module is configured to adjust the cognitive stimulation game therapy by adjusting one or more game parameters, game dosage and game timing, and is configured to adjust the guided learning therapy by adjusting the learn amount and timing, and learning content, and is configured to adjust the reminiscence therapy by adjusting the timing of content, content topics and stimulus and is configured to adjust the physical and mental wellness therapy by adjusting a modality, an intensity and a duration of physical exercise or mental exercise.

In one form, the digital cognitive therapy delivery module provides a user interface on a computing device comprising:
a reminder and calendar module configured to allow patients to record reminders and to notify the patient of a scheduled therapy, and monitors therapy compliance;
a note module configured to track goals and record electronic information to assist with daily living activities;
a medication schedule module configured to record a medication schedule and track compliance;
an electronic gratitude journal;
a mood tracker configured to estimate and/or record a mood of a patient, and to provide feedback on past mood history and to provide mood data to a clinician and/or the cognitive analytics engine;
a social media module to facilitate communication with family, friends and support groups;
a gamification system which awards points for completion of therapy or tasks, and rewards for achieving specific points goals, and a comparative score based on treatment progress with respect to other patients with similar diagnosis;
a diet tracking module configured to collect consumption data and provide dietary recommendations; and
a therapy module configured to provide the plurality of therapies to the patient.

In one form, the method is implemented using a cloud computing platform and the digital cognitive therapy delivery module is configured to execute on one or more patient mobile computing devices.

According to a third aspect, there is provided a computer readable medium comprising instructions for causing a processor to implement the method of the second aspect.

### BRIEF DESCRIPTION OF THE DRAWINGS

Embodiments of the present disclosure will be discussed with reference to the accompanying drawings wherein:

FIG. 1 is a schematic diagram of the system architecture of a computational personalized cognitive therapeutic system according to an embodiment;

FIG. 2 is a schematic diagram of a digital cognitive therapy delivery module according to an embodiment; and

FIG. 3 is a flow chart of use scenarios of the system according to an embodiment.

In the following description, like reference characters designate like or corresponding parts throughout the figures.

### DESCRIPTION OF THE INVENTION

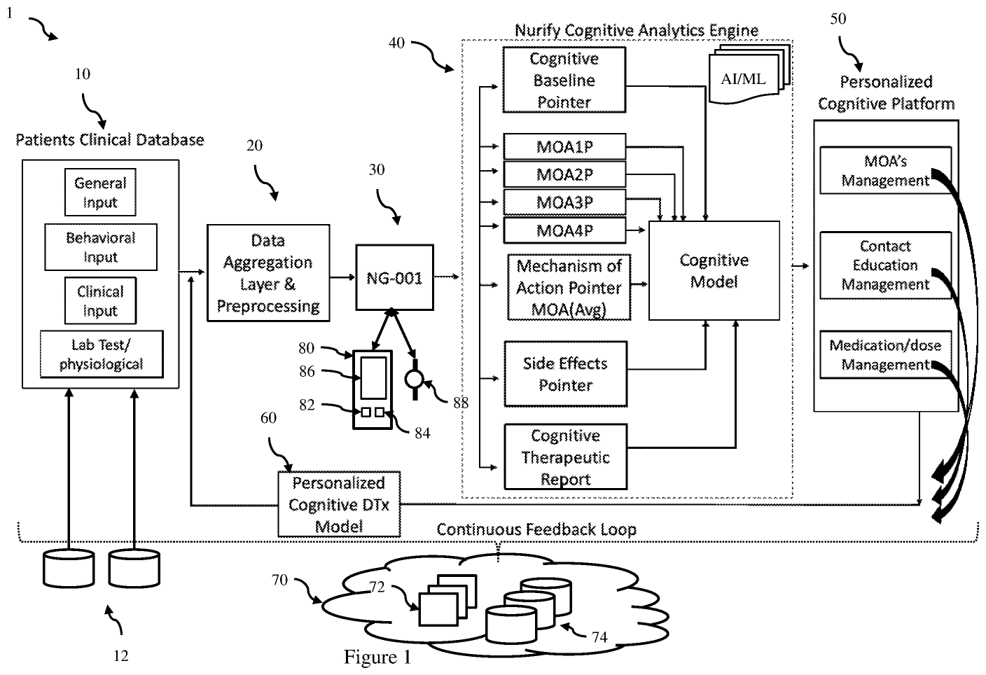

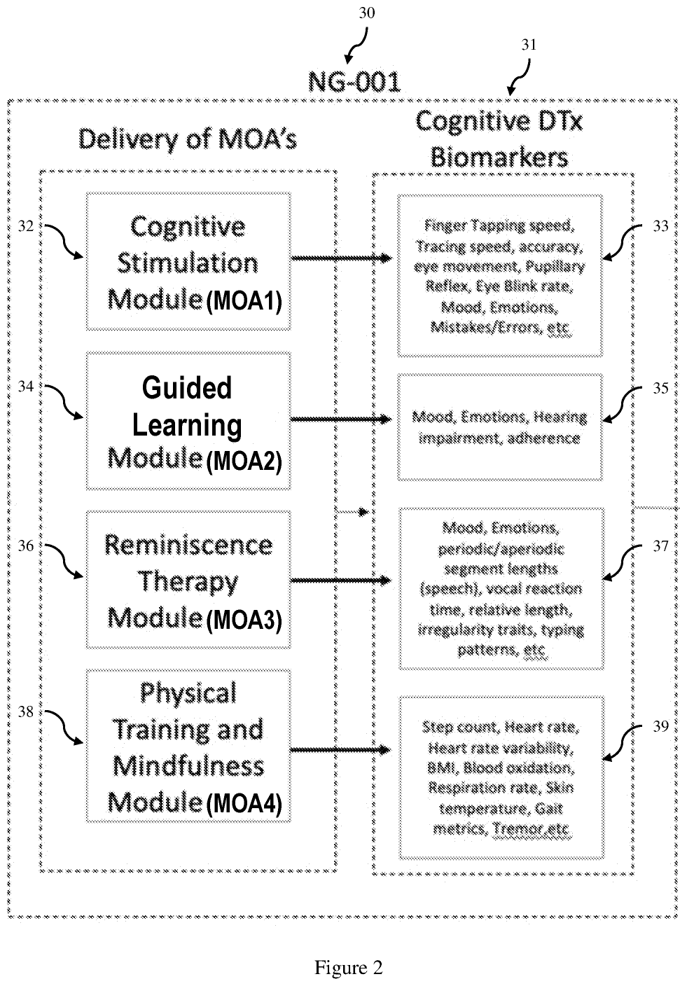

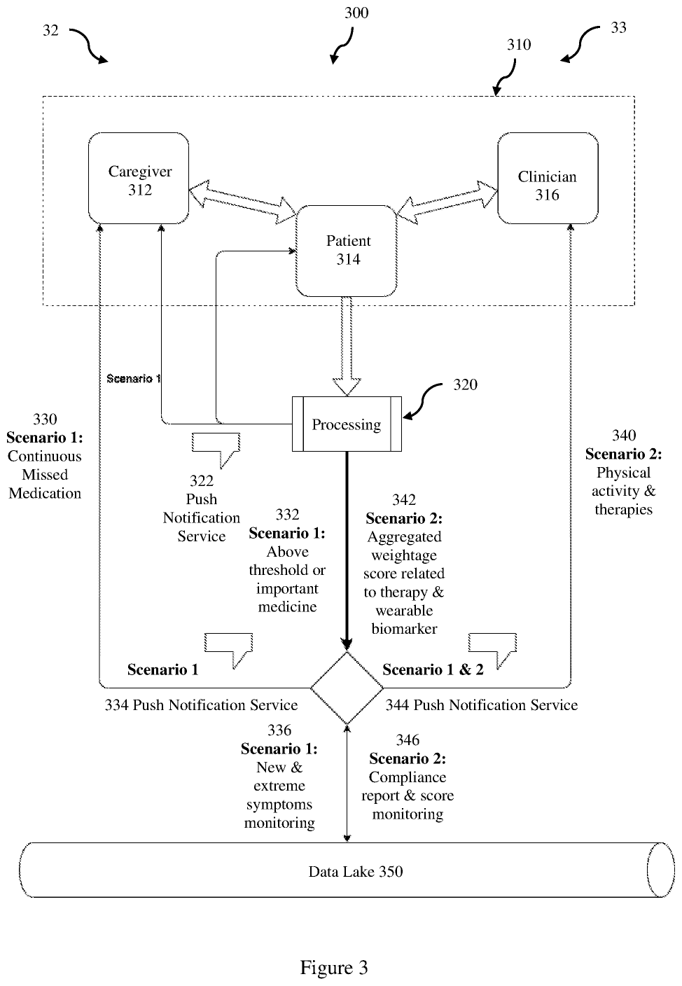

Referring now to FIG. 1, there is shown a schematic diagram of the system architecture of a computational personalized cognitive therapeutic system 1 according to an embodiment. FIG. 2 is a schematic diagram of a digital cognitive therapy delivery module 30 according to an embodiment and FIG. 3 is a flow chart of use scenarios of the system 1 according to an embodiment.

Embodiments of the system are designed to either maintain or improve cognitive functioning in patients with either Mild Cognitive Impairment (MCI), Alzheimer's Disease (AD), prodromic AD, or other neurodegenerative disorders. The system may also be used by healthy elderly to maintain or improve their cognition. The goal in particular is to improve or maintain processing speed, executive function, attentional control, perception (across all senses), sustained alertness, metacognition, immediate and delayed memory (short-term and long-term), decision making, emotional control, overall cognitive functioning, and daily activity functioning. Apart from targeting cognitive health, use of the system may result in improved physical fitness, psychological wellbeing (mood) and quality of life (QoL) in MCI, AD and other patients with neurodegenerative disorders. The system also can improve the wellbeing and QoL of caregivers, and help clinicians provide more timely and appropriate healthcare.

Embodiments of the system comprises a patient clinical database 10, a data aggregation layer and data pre-processor module 20, a digital cognitive therapy delivery module 30, a cognitive analytics engine a personalised cognitive platform 50 configured to use the plurality of metrics to personalize the personalised cognitive digital therapy model 60. The system may be implemented on a computing system including a cloud computing platform 70, a plurality of mobile computing devices 80, wearable computing and medical devices 88, and may be configured to interface with medical devices, laboratory management systems and electronic health records. The cloud computing platform may comprise one or more processors 72 and one or more memories 74, and the mobile computing devices 80 including one or more processors 82, one or more memories 84 and one or more input/output devices 86 such as a touch screen display, microphone, and speakers, wearable computing devices. These may network and communicate with wearable devices, medical devices and other computing systems such as laboratory management systems and electronic health records.

The system 1 is configured to personalize digital therapy by focusing on a range of data points which include medication that the patient is on, the dosage of the medication, side effects of the medication, behavioural changes due to multiple digital therapies with different mechanisms of action (MOAs) measured using a combination of digital cognitive biomarkers, behavioural and physiological biomarkers, and reported outcomes by the patient. Leveraging the known information of the therapies, the system can personalize a personalised cognitive digital therapy model 60 for each patient to address specific treatment targets for a patient and maximize the anticipated positive effect from the treatment. The model may be reviewed and updated by the clinician through a clinician platform or user interface when there is a change (or suggested change) in the therapy, including medication and/or dosage, or when expected positive and/or side effects are updated.

The patient clinical database 10 comprises a data acquisition interface configured to receive and store input data for a plurality of patients from a plurality of heterogeneous data sources 12. The input data sources for a patient comprise one or more wearable devices and one or more of an electronic medical record, a digitised caregiver record, a laboratory information management system, a clinical database, and/or a patient on-boarding module.

The input data for a patient comprises general input data such as personal particular and sociodemographic data; patient medical history and background health data; medication data; clinical data; and wearable data comprising behavioural data and physiological data. As shown in FIG. 1, this input data can be grouped into general input data, behavioural data, clinical input data and laboratory test/physiological data. The input data may comprise static data such as personal particular and sociodemographic data on the patient and patient medical history and background health data including personal and family health history and risk factors (smoking, alcohol, diet, etc.). Other data may change over time or be added or updated over time, such as medication data, and data reports such as laboratory results, diagnostic reports, and physiological data from wearable devices and medical devices. As will be described below, the system generates a personalised cognitive digital model 60, including personalized model outcome and treatment parameters used to adjust (or titrate) the individual's therapy. The patient clinical database 10 may be used to store the treatment parameters generated from the personalised cognitive digital model 60.

The general patient input data includes personal particular and sociodemographic data and include data points captured from the patient and/or caregiver during initial on-boarding of the patient. This includes, but is not limited to age, sex, current location, personal particulars (Name, DOB, Place of birth, Native language), education level attained, work profession (Role, location, years, etc.), Hobbies, Family history (dementia/Alzheimer's) and current medications. Behavioural interaction data comprises parameters which are captured during initial on-boarding, and during subsequent use of the system, from the patient and/or caregiver and includes, but are not limited to, a patient's preferences in different aspects of life, disease trigger points if any, hobbies, alcohol consumption, smoking, diet, etc. Medication data includes both the current drugs and scheduled dosage prescribed to a patient along with other co-morbid conditions, medication side effects, family history for Alzheimer's Disease and Dementia, and other medications and dosage for other conditions. Laboratory test data and physiological input data includes data from Electronic Medical Records (EMR) or Patient/caregiver reported data including current health status/diagnosis, BMI, Heart rate, Respiration rate, medication history, diagnostic report (MRI, blood test reports, paper-based tests, etc.), interpretation from clinicians, and other medical information. The results of paper based tests performed by clinicians like MMSE, CDR-Cog13, ADCS-ADL test are also fed into and stored in the patient clinical database 10.

The data aggregation and pre-processing module 20 is configured to aggregate and pre-process the input data in the patient clinical database and generate a patient profile for each patient (which may then be stored in the patient clinical database 10). The patient profile is used to generate the personalised cognitive digital model 60.The patient profile is updated when there are changes in the inputs from the patient, caregivers, and clinicians (e.g., updated MRI reports, lab tests, screening tests etc.). Individual patient profiles serve as a baseline for further monitoring and assessment of the effectiveness of treatment.

As the input data is obtained multiple different or heterogeneous sources, the data aggregation and pre-processing module 20 processes the data using several methods such as data cleansing, dimensionality reduction and data transformation to prepare the data for further analysis and use by the cognitive analytics engine 40 (and in particular the AI/ML models). This ensures the AI/ML models are trained and use high quality data (or at least consistent data)and can also be used to obtain/discover preliminary ‘knowledge’ for the profiling and further analysis.

Data cleansing comprises cleaning the data for erroneous, missing, redundant, noisy and outliers to ensure the overall data's consistency, reliability and accuracy. In the situation of erroneous and missing features, the entire datapointhuple' may be removed, or a standard/nearest/statistical measured value (or central tendency measure) used to fill in the data depending on the distribution of the collected datasets. Similarly, other algorithms/methods are implemented for data cleaning depending on the required datapoints and their issues. Dimensionality reduction is used to remove redundant and unnecessary features from the dataset. For reduction, filters (such as based on variance and correlation) are applied along with other feature selection techniques, where necessary and relevant features are identified, and if required, using the selected existing features, pertinent new features are derived, which is termed as feature engineering. With filters, the correlation between the features is plotted and checked for the relevant or redundant features. Depending on the distribution and types of the features in the input data, matrix factorization (such as PCA), manifold learning methods (like t-SNE) maybe applied. Data transformation is performed to normalise the data and correct for effects due to use of heterogeneous sources. This may include the use of data standardization and data normalization methods, and automated conversions to the same measuring units.

A digital cognitive therapy delivery module 30 is configured to deliver a plurality of therapies according to a personalised cognitive digital therapy model 60 to a patient using one or more computing devices 80, 88. Each therapy has a different mechanism of action (MOA) and comprises a plurality of adjustable parameters. Additionally the digital cognitive therapy delivery module is further configured to collect a plurality of digital cognitive biomarkers 31 for each therapy, and a plurality of behavioural and physiological biomarkers from one or more wearable devices 88 and/or the one or more computing devices 80. The computing devices are also used to measure interactions of the patient with the digital cognitive therapy delivery module, such as by capturing audio and video of the patient whilst performing activities.

In one embodiment the therapies comprise at least a cognitive stimulation game therapy 32 (MOA1), a guided learning therapy 34 (MOA2), a reminiscence therapy 36 (MOA3) and a physical and mental wellness therapy 38 (MOA4). Other therapies with other MOAs may also be provided by the digital cognitive therapy delivery module 30. The specific modules used for therapy can be chosen based on what disorder is being treated. The system can further refine the recommendation based upon which cognitive abilities are impaired. For example, if a person shows more impaired memory they may need more cognitive games designed to improve memory as well as guided instruction in how to augment their memory using the calendar module provide by an application (App) on the user device 80.

The cognitive stimulation game therapy module 32 (MOA1) comprises cognitive brain training games which are designed to drive outcomes and focusing on areas like but not limited to memory, logical reasoning, perception, attention, coordination, etc. Each cognitive game is designed to train different cognitive domains with the hypothesis that performance in that cognitive domain will improve with training These include visual perception, visual search, attentional control, selective attention, short-term memory, long-term memory, executive function, navigational ability, language, multitasking and metacognition.

In one embodiment the cognitive game is “Family and friends recognition training”
The user can add in photos/videos either from their own camera, or submitted by the care partner or friends. The photos can be labelled with names and information about who is in the photos, their relation to the patient, and other interesting facts about the people in the photos.
The system will then show them the videos/content and ask them to match the names or information.
The accuracy of the user will determine how often a particular person is displayed, with lower accuracy items being displayed more often, and less recently shown items first. The system can also use the principle of expanding retrieval practice to show users photos of successfully recognized photos/videos with increasing delays to retain memory.

The progression of each user through the levels can indicate their learning ability. Learning ability may vary by domain The speed they progress through levels may also be used to model what scores they would get on standardized neuropsychological tests. Types of errors and reaction times as well as other datapoint can be used to predict neuropsychological test scores.

The obstacles and manipulations in the game can be made to vary so that they appear at multiple variations within the same level (e.g. an obstacle may approach the user at several different speeds). This will allow a model to be made to determine what effect each manipulation has on performance

Within a game, a model can be made to determine what aspects of the game are leading to the user scoring lower points, or progressing more slowly towards the goal point of the game. This model can then be used to adjust the obstacles that are causing difficulty for the player. (E.g. if a user is having trouble doing complex visual search, the items to search for can be adjusted to be more distinct from the items to ignore, or if the user is reacting too slowly to avoid a certain obstacle, the speed can be individually adjusted for that obstacle).

The model can also predict which items in the game are not causing any difficulty or leading to any mistakes and adjust the difficulty to be higher.

After the adjustment, the model will then be computed again and other items can be adjusted in the game so that the user is continuously kept at a level of difficulty that is ideal for learning.

The rate of adjustment can be varied and then the learning rate can be compared based on the rate of adjustment. Thus, the rate of adjustment can be optimized so that it leads to the best learning rate for the individual.

The digital cognitive therapy delivery module 30 monitors game play to collect a plurality of digital cognitive biomarkers for the cognitive stimulation game therapy 33 comprising game specific performance, finger tapping and finger movement related biomarkers, reaction time between a stimulus exposure and a response, and a proxy index of a cognitive load. These biomarkers 33 may be calculated from measurements such as finger tapping speed, accuracy, eye movement, pupillary reflex, mood, facial expressions, mistakes/errors, etc.

Game-specific performance biomarkers include overall game scores and the differential within-game performance/accuracy relative to gameplay parameters. For example in a runner game, success in passing obstacles depending on type and grid of the obstacles is measured.

A range of finger tapping and finger movement related biomarkers may be calculated. In one embodiment screen coordinates of finger tapping touchpoints and swipes are collected from the cognitive games. The spatial distribution of these point patterns and/or movement patterns are then analysed to estimate the perception and motor attentional processes of a patient (concentration/diffusion of motor and sensory based attention; how well a patient operates with each of the arms, including fingers, hands, wrists, elbows, etc.). Additionally or alternatively tapping speed, acceleration and accuracy (including precision) are recorded. Finger dexterity may be estimated from tapping accuracy and measures such as the mean distance between closest point pairs to mean distance between all point pairs, and the mean distance between consecutive points to mean distance between all point pairs. The ability to undertake skilled sequences of movements in response to instructions and stimuli presented to the user may also be collected and uses to assess physical and cognitive capabilities of the patient.

Biomarkers measuring the reaction time between a stimulus exposure and response biomarker may be calculated by recording timestamps of diverse game stimulus and player actions. Timestamp-based differences may be computed between sequences of stimulus and responses allowing an estimate of a patient's processing and reaction speed. The number of stimuli that can be successfully tracked at the same time and responded to correctly may be measured as well as a complexity of stimuli that can be tracked.

A proxy index of cognitive load biomarker may be obtained from wearable sensor data including blood volume pulse (HRV) and skin conductance. The biomarker is designed to record performance on different game aspects and related error types linked to cognitive demands (immediate and delayed memory, executive functioning, perception, etc.). A range of manipulations to cognitive games to vary cognitive load and demands may also be performed and tracked.

In the guided learning therapy module 34 (MOA2), also referred to as the Cognitive rehabilitation module, educational content is developed to help the patient anticipate and prepare for future issues they may encounter. This module includes a focus on Activities of Daily Living (ADL), and Instrumental Activities of Daily Living (IADL). These are divided into basic and instrumental activities and this module educates the patient in performing these tasks. Some examples are (but not limited to) information about neurodegenerative disorders and their progression, maintaining family, friend, and partner relationships, financial planning, home safety, dealing with depression and apathy, and how to stay motivated and live a fulfilling life. This may be provided in the form of therapy guided learning and self-guided learning. Data such as mood, emotions, comprehension of the therapy by questions related to application or recollection, hearing impairment, and adherence may also be measured when using the module.

Therapy guided learning is provided as comprehensive mini-courses, in which the patients are given short videos and texts to prepare them for future issues that may develop, and to prevent accidents or bad health incomes. The time spent watching the videos is tracked and can be used to track sustained attention, memory, interest, and cognitive state. A short quiz at the end of each lesson can be used to assess retention of the material. Changes in scores over time can be used to track cognitive change, and alert the caregiver or clinician. There are many educational topics available, and they can be customized to best match the patient's needs depending on the disorder and their current living situation (e.g. they live with their adult children, they live with a healthy partner, or they live alone, the amount of resources they have available). This customization can occur using an algorithm and may also be directly by clinicians to cover unique cases that cannot easily be covered by the algorithm. The customization by the clinician can then be used to train the algorithm to learn to suggest new educational content for other patients.

Self-guided learning allows users to select additional educational material. This enables them to find material that is relevant to their current situation, or interest. The users can also directly enter questions, and then the material can be updated directly in the system with additional relevant timely news for the user. New posts can also be updated from time to time without requests from the user, but instead due to new discoveries, or by using data from reading of other topics to determine what is of most interest to users. These new posts can be displayed in a way to catch the user's attention.

The digital cognitive biomarkers for the guided learning therapy 35 comprises quiz results, answer confidence, speed of information processing of content, or a time spent with content.

The reminiscence therapy module 36 (MOA3) focusses on reminding the patients regarding their past happy moments and showing them pictures/videos and asking optimized questions related to the same. Data on mood, emotions, speech, vocal reactions, typing patterns, etc. may be collected and analysed to derive biomarkers.

For specific topics, guided questions elicit further feedback so that patients will be able to continue even if they initially run out of material. The addition prompts to elicit further discussion can be displayed when the individual has paused for a set amount of time (or as indicated by the voice pattern that they are stopping), or other indications such as negative moods so that new topics can direct the user towards a better mood or cognitive state.

This module is also configured to train multiple levels of memory systematically, so that the entire memory system can work better together. This may comprise
Very recent past recall of something that occurred within a few hours or at most a few days (e.g. “What is something enjoyable you did today?”).
Far recall of specific questions about early in their life (e.g. “Tell me about the house you grew up in.”). This could easily be more than 7 or 8 decades ago for the patients.
Future projection—(e.g. “Where would you like to travel to next and what would you like to do there?”). There is evidence that the same system used to recall past information is also used to simulate possible future events. The reason is that to predict the future, one must have a history of past experiences to draw upon to simulate what could happen. This exercise helps them practice utilizing this part of the memory system.
Picture based recall—Pictures are either period photos tailored to the patient's past experience (e.g. the place where they grew up, or have spent much of their life, or specific items or events that they would have witnessed either in person or through news and media), or are contributed by the caregiver so that they are personal memories.

This leads to emotional well-being by coming to terms with the past, and integrating past existence with the present and also leads to positive emotion by allowing the patient to relive happy memories.

The digital cognitive biomarkers for the reminiscence therapy 37 comprise a language marker derived from textual analysis or audio analysis, and a speech characteristic derived from audio data of the patient. The language text markers may include:
Positive/negative sentiment ratio from dictionary-based sentiment analysis;
Semantic graph features (key concepts); and
Vocabulary and syntactic indicators—complexity/diversity/length/grammatical agreement (case marking, conjugation, correct pronoun use, etc.).

The speech characteristics biomarkers may include all of the language markers above plus:
Acoustic voice characteristics (volume, pitch, speed); and
Phonemes (speech) and silence (non-speech) segments length and periodicity, stress patterns within both words and sentences.

The physical training and mindfulness module 38 (MOA4) focusses on physical wellness such as exercises and mental wellness, such as meditation and mindfulness modules designed to drive outcomes in the patient.

In relation to physical wellness, a series of exercises or activities may be prescribed. A wearable device 88 can be used to provide data such as heart rate, heart rate variability, galvanic skin response, and gyroscope which can be used to determine effort and difficulty for that individual during exercises. This information is then used to determine whether the exercise should be kept the same, or changed to either less or more demanding. The exercise difficulty can be changed by duration of the exercise, the type of movements, the speed of the movements, or the amount of weight used, etc. The data from the wearable can also be used to determine physical strengths and weaknesses of the individual, so that the exercise can be customized to best treat that person. The goals of the physical exercise can be chosen by the system, or by the physician. For example, someone who is not very good at walking could be given progressively longer walks until they reached a set level that is deemed acceptable for independent daily functioning. Another example is a patient who cannot lift very much and thus is given exercises to increase upper body muscle strength. Other exercises could address stability and core strength. An additional example could have to do with flexibility, and focus on increasing stretching ability.

In relation to mental wellness activities such as meditation and mindfulness activities may be prescribed. A wearable device 88 can provide data such as heart rate, heart rate variability, galvanic skin response, and gyroscope which can be used to determine how well the mental exercises are at reducing stress and at capturing the focus of the user. An electrode attached to the user's head could also record electroencephalographic (EEG) information that could determine whether the user has entered a relaxed mental state. This information can be used to adjust the type of mental exercise given to the user. For example, if the heart rate of the user and galvanic skin response is decreasing this may indicate the user is becoming more relaxed, and thus less instruction needs to be given to the user, but if the user is becoming more stressed and heart rate is not decreasing, the user may need additional guidance. This can be continually adjusted during a single session, so that whenever the heart rate increases an intervention can be done to relax the user. Mental exercises could include several different types of meditation and mindfulness, including but not limited to body scan, focused meditation (by focusing on one object such as breathing), unfocused meditation, focusing on a mantra, mental imagery, compassionate meditation, or other approaches. Alternatively or additionally direct feedback may also be obtained from the patient, or a video may be taken of the patient whilst they are doing the exercise (for example using a camera on a user computing device 80) which is then analysed or reviewed. Based on the analysis the type of activity they are prescribed can then be adjusted.

The plurality of digital cognitive biomarkers for the physical and mental wellness therapy 38 comprises the access frequency of content, a time spent with content, an application (App) opening and closing frequency, a task completion within an allotted time, and a compliance with an allotted task, a direct feedback from the patient on the likeability and difficulty of the therapy via a questionnaire, and an emotional expression capture indicating a level of enjoyment.

The digital cognitive biomarkers 35 for each therapy, and behavioural and physiological biomarkers enable continuous monitoring and diagnostic assessment of patients' health and psycho-cognitive functioning. The system utilizes digital phenotyping methods and extensive set of digital cognitive biomarkers based on different types of input data including (1) data generated from “user-system interaction” when using digital therapy modules (MOAs) (e.g. digital game data, finger movement data, audio recordings, eye tracking, facial expression monitoring, photo and video submissions) and (2) passively collected behavioural and physiological data from a wearable device. The set of cognitive, behavioural and physiological digital cognitive biomarkers capture and characterize: performance under different cognitive demands, physical activity, sleep duration (time in bed), circadian rhythms, autonomic nervous system functioning and sympathovagal balance, physiological stress and arousal, proximate cognitive load, depressive mood, inflammation markers, compliance to wellness exercises, speech patterns, sentiment and semantics, etc.

Behavioural and physiological biomarkers may also be collected from one or more wearable devices 88, medical devices, and user computing devices 80. These biomarkers may comprise one or more of a movement biomarker obtained from an accelerometer and/or gyroscope (including a gyroscope and a magnetometer), an electrodermal activity or skin conductance biomarker (time- and frequency-domain features), a photoplethysmography or blood volume pulse biomarker, a heart rate biomarker, a heart rate variability biomarker, a skin temperature biomarker (time-domain features), finger tracking systems to assess finger dexterity and cognitive load, eye tracking to monitor pupil size, eye movements, area of focus, facial expression recognition systems (e.g. camera based systems) including recognition systems configured to estimate mood, and electrophysiological measures that serve as indices of neural activity. These may also include HRV metrics (time- and frequency-domain features including but not limited to SDNN, RMSSD, SDSD, pNN25, pNN50, Poincare plot descriptors SD1 and SD2, HF-HRV, LF-HRV, VLF-HRV, etc.), Sleep duration (bed in time), overall daily physical activity, daily time spent in different intensities of physical activity, sedentary time and circadian rhythm metrics (nonparametric and cosinor-based including but not limited to interdaily stability, intradaily fragmentation, relative amplitude, rhythm autocorrelation, interdaily coefficient of variation, mesor, amplitude, acrophase, alpha and beta coefficients, pseudo F-statistic). These may be used for general patient monitoring as well as for generating digital cognitive biomarkers for the physical training and mindfulness module 38 (MOA4).

A range of digital cognitive biomarkers will be derived from the wearable device data including digital cognitive biomarkers characterising motion and physical activity, sleep duration and sleep quality, circadian rest-activity and metabolic rhythms, autonomic nervous system functioning and sympathovagal balance, physiological stress and agitation (emotional arousal), mood symptoms (depressive symptoms), neural activity, and/or a proxy index of cognitive load. Wearable-based digital cognitive biomarkers will characterise time periods of different lengths (from 5-minute intervals and up to weeks) due to different methodologies and aggregation scales behind them. For patient monitoring and treatment response assessment the data flow may be as follows:
Acquiring input data from a wearable device (e.g. acceleration, skin conductance, skin temperature, blood volume pulse, electrophysiological measures that serve as indices of neural activity), or user computing device (e.g. touch screen for finger movement, camera with eye tracking or facial recognition systems);
Detecting missing data, data resampling and interpolation;
Splitting data into non-overlapping sliding windows (5-minute intervals by default);
Applying specific biomedical signal processing procedures including filtering, signal decomposition, smoothing, and data transformation depending on the type of input signal;
Feature extraction using time, frequency and time-frequency domains analysis and other dedicated methods—extraction of aforementioned digital cognitive biomarkers for corresponding periods of time;
Continuous monitoring of digital cognitive biomarkers dynamics and anomaly detection. This may include looping digital cognitive biomarkers dynamics to the automated personalization platform and generating summary reports for the clinical care team.

A cognitive analytics engine 40 is configured to process the patient profile, digital cognitive biomarkers, and the behavioural and physiological biomarkers using an ensemble of population-based and personalised prediction models trained using a plurality of Artificial Intelligence (AI) and Machine Learning (ML) methods. The cognitive analytics engine 40 is configured to generate a plurality of metrics to characterize a current cognitive (or psycho-cognitive) state of the patient and estimate the potential future improvement comprising the probability and size of an expected effect. The plurality of metrics may comprise a cognitive baseline pointer, a mechanism of action pointer for each of the MOAs, an MOA Average pointer and a side effects pointer. A cognitive therapeutic report may also be generated. These metrics may be generated on demand or at least once per day or other periodic intervals. For example a table (or similar data structure) of normed performance data may be collected/extracted for similar patients controlled for age, education, career, socio-economic, and, physiological, health and historical factors. This could be used by the AI/ML to determine how the performance of the patient compares to similar (matched) patients to determine how their state compares to what their maximal or minimal predicted cognitive range would be. For example, an 80 year old would not be expected to be able to respond as quickly as a 50 year old (indicating a slowed processing speed), and may also be expected to recall fewer details (indicating less precise memory). However by first selecting a matched patient cohort, the relative performance of the patient can be assessed and used to make predictions of expected progress (so treatment can be adjusted if it fail to meet expected progress). The cognitive analytics engine 40 may be configured to create a matched patient cohort which is used to train an AI/ML models used to assess the patient state/relative performance, or the matched patient cohort may be used to generate a matched patient distribution which the cognitive analytics engine 40 uses to assess the patient state/relative performance. In another embodiment the cognitive analytics engine 40 may be configured to periodically compute a set of AI/ML models each with an associated patient cohort. In this embodiment the cognitive analytics engine 40 generates multiple groups of similar patients using the data in the patient clinical database 10 (i.e. generate multiple patient cohorts), and then generate a AI/ML assessment model for each group/patient cohort to generate a set of AI/ML models. During analysis of data from the digital cognitive therapy delivery module, the cognitive analytics engine 40 then matches the patient to one of the patient cohorts, and uses the associated AI/ML model for that group to assess the patient state and performance In some embodiments the cognitive analytics engine 40 may estimate the specific side effects or combination of impairments the patient is suffering from and generate or select a cohort of patients with similar side effects or impairments. This may be used to assess the relative state/performance of the treatment or to identify potential treatments based on identifying the treatments common to the top responding patients in the cohort, e.g. by using a quantile regression or similar model to compare the treatments of the top 10% vs the middle 50% of patients. In some embodiments the cognitive analytics engine 40 may use AI/ML models may use the digital, behavioural and physiological biomarkers to identify the specific side effects the patient is suffering and thus determine appropriate treatments which compensate or counteract for these side effects. Regeneration of the set of AI/ML models may be done periodically as new patients or new performance data is obtained. In some embodiments multiple AI/ML models may be used to assess performance or state of the patient. These multiple models may be generated using different training datasets, parameter sets or model architectures, each of which generates a prediction, and ensemble methods then used to combine results.

The cognitive baseline pointer is an estimate of a change in the cognitive state of the patient with respect to a baseline generated using the patient profile and an expected behaviour effect on the patient generated by the cognitive analytics engine. That is given the input patient profile (or most recent updated patient profile) and expected effects on the behaviour of the patient, the cognitive baseline pointer derives the current state the patient is at and how the baseline is improving from previously delivered therapies to the subsequent therapies.

The mechanism of action pointers for each of the mechanisms of action (MOA1P, MOA2P, MOA3P, MOA4P) each estimate an effect level with respect to an expected effect level for the associated therapy generated by the cognitive analytics engine. That is given the expected effects on the behaviour, MOA1P, MOA2P, MOA3P, MOA4P are real-time individual measure the effectiveness of the digital cognitive therapy which measure how far the therapy meets the expectation (as generated by an AI/ML model). The greater pointer, the greater the positive influence or impact of the therapy. Relationships between pointers and the treatment effect will be further personalized and the unique set of pointers for each patient will be compiled. Pointer examples include:
MOA1P—Game-specific and differential within-game performance
MOA2P—Quiz results and patient's video and content interaction behaviour
MOA3P—Vocabulary, syntactic, semantic, prosodic, phonological, and sentiment features of speech and written texts
MOA4P—Level of relaxation measured with HRV dynamics

The average mechanism of action pointer (MOA Avg) estimates an average effect of the plurality of therapies with respect to an estimate effect generated by the cognitive analytics engine. That is given the expected effects on the behaviour, MOA Avg is a real-time measure of the effectiveness of the digital cognitive therapy which means how far the therapy meets the expectation. The greater pointer, the greater the positive influence or impact of the therapy.

The side effects pointer measures a severity of one or more side effects. The side effect can be known or even unknown and the side effects can be measured by but are not limited to real-time physiological parameters, patient behaviour, patient-reported symptoms, and optimized questionnaire or lab reports. The greater SEP, the worse the side effects.

A Cognitive Therapeutic Report (CTR) may be generated. The CTR is a daily summary report of the effect of the therapy. CTR can measure the therapy effectiveness and quality of life of the patient.

The metrics generated by the AI/ML models together with the cognitive therapeutic report, proposed treatment and medication adjustments, and estimated effect levels are combined into a cognitive model which characterize the current cognitive state of the patient and estimate the potential future improvement comprising the probability and size of expected effects through adjustment of digital treatments and medications.

A personalised cognitive platform 50 is configured to use the plurality of metrics (or cognitive model containing the metrics) to personalize the personalised cognitive digital therapy model 60 for each patient by adjusting one or more of the plurality of parameters for one or more of the plurality of therapies to maximise the estimated effect level, and to use the metrics to generate one or more alerts if a therapy does not meet an expected threshold effect level or a side effect exceeds a threshold side effect level to enable adjustment of a medication by a clinician. These may be automated changes or may be reviewed and authorised by a treating clinician.

The system thus iteratively refines the personalised cognitive digital therapy model 60 for each patient over time by selecting specific therapies from the plurality of therapies and adjusting the associated adjustable parameters, and obtaining an estimate effect of the adjustments, and after delivery of an adjusted treatment by the digital cognitive therapy delivery module 30, the cognitive analytics engine generates the plurality of metrics to assess actual effects compared to estimated effects in order to further refines the personalised cognitive digital therapy model 60 by the personalised cognitive platform 50.

The personalised cognitive platform 50 may comprises a MOA management module, an educational content management module and a medication/dose management module. The MOA management module may use at least the mechanism of action pointers and the average mechanism of action pointer to adjust one or more of the parameters for one or more of the digital therapies and to adjust the dosage of each of the plurality digital therapies to maximise the estimated effect level of a therapy. The content education module is configured to adjust a digital content provided to a patient based on the patient's interaction behaviour measured by the digital cognitive therapy delivery module. For example, a discover section comprising videos and articles could be monitored and the content updated based on user selections and activity. The medication/dose management module is configured to record clinical data including medication and dosages and to generate suggested changes to medication and dosages using at least the side effects pointer and the cognitive baseline pointer. This may also include changes to behaviour modification/change programs (e.g. smoking cessation, fear response), as well as suggested healthcare appointments, investigations, lab tests, hospitalization, etc.

In a further form, the MOA management module is configured to adjust the cognitive stimulation game therapy by adjusting one or more game parameters, such as for addressing a particular gameplay aspect which appears as a ‘cognitive treatment target’ for improving specific cognitive abilities (for example, sustained attention, or ability to retain more items in memory) or which gives most difficulty to patients, along with game dosage and game timing. The MOA management module is further configured to adjust the guided learning therapy by adjusting the learn amount and timing, and learning content, and is configured to adjust the reminiscence therapy by adjusting the timing of content, content topics and stimulus and is configured to adjust the physical and mental wellness therapy by adjusting a modality, an intensity and a duration of physical exercise or mental exercise.

As treatment progresses the dosage (or balance) of different treatments will change and be refined to best suit the patient's progress and needs. The feedback process is ongoing, with repeated cycles of digital treatment using the digital cognitive therapy delivery module (according to the current personalised cognitive digital therapy model), evaluation by the cognitive analytics engine, and adjustment of the personalised cognitive digital therapy model by personalised cognitive platform to refine and improve treatment. The metrics generated by cognitive analytics engine allow evaluation of treatment progression and selection of appropriate digital therapies to improve treatment (including which specific therapies. In some cases specific digital therapies may be omitted for a period of time, or only used intermittently to allow treatment to focus on other digital therapies for which there is a greater need. For each digital therapy there will be a range of individual treatments and the personalised cognitive platform is configured to select which of these individual treatments to prescribe as the personalised cognitive digital therapy model. For example cognitive stimulation game therapy comprises a library of different games with a range of different adjustable parameters. These games will capture a range of digital cognitive biomarkers, with some games capturing more digital cognitive biomarkers and other capturing less. When personalising the personalised cognitive digital therapy model, specific games are chosen and the values of associated parameters selected. The digital cognitive biomarkers to be collected may also be selected, or default biomarkers may be stored and used for each specific game. The cognitive analytics engine may also be used to predict the expected effect level, which can then be assessed against the measured performance (via the digital cognitive biomarkers) after the next set (or multiple sets) of treatments. For example if a patient has shown good progress or capability in the reminiscence therapy but is showing signs of possibly depression, then future treatments (as specified in the personalised cognitive digital therapy model) could focus or emphasise (i.e. up the dosage) of physical and mental wellness therapies. Alternatively if the patient is showing signs of physical weakness and signs of cognitive impairment, the dosage of the different therapies could focus on cognitive stimulation game therapy, and physical and mental wellness therapy (at the expense of guided learning therapy and a reminiscence therapy).

The cognitive analytics engine 40 will generate alerts which are sent to the personalised cognitive platform 50 for review by clinicians and caregivers. These alerts are generated with explanations when the effect of the therapy does not fulfil the expectation or there are severe side effects.

The personalised cognitive platform 50 assists clinicians and caregivers in monitoring and managing patients after the patients have taken digital therapy, medications, enabling further interventions, as well as titration of the medications and therapy and therefore help the patients to improve their health and daily living. The titration of the therapy and medication will be done remotely by the clinicians via a user interface depending on the feedback obtained from cognitive analytics engine.

In one form, the digital cognitive therapy delivery module in addition to providing a therapy module configured to provide the plurality of therapies to the patient as described above, may also provide additional modules for the patient. These may be provided via a user interface or an application (App) on the computing device 80. These include a reminder and calendar module, a note module, a medication schedule module, an electronic gratitude journal, a mood tracker, a social media module, a gamification system and a diet tracking module.

The reminder and calendar module configured to allow patients to record reminders and to notify the patient of a scheduled therapy, and monitors therapy compliance. This is electronic calendar/reminder system can be used by patient as an external memory to supplement their own internal memory. It can also be used to schedule when therapy will occur and helps maintain therapy compliance.

The note module is configured to track goals and record electronic information to assist with daily living activities. Patients can keep track of goals they'd like to complete which have not yet been scheduled for a specific time. They can also add photos or any information (such as web links) to this electronic scrapbook to remind them of anything like where specific items are held, how to use electronic appliances, recipes, motivational quotes, etc.

The medication schedule is configured to record a medication schedule and track compliance. This can be linked up with an electronic pill detection system either from the medical dispenser, or tracking physical consumption of the medicine using e-pills or similar technology.

The electronic gratitude journal can be used help the patient appreciate small things that are going well in their life instead of dwelling and ruminating on the negative. It can consist of guided questions to direct the focus of the user to come to terms with the experiences and evaluate them using a more positive/productive viewpoint.

The mood tracker is configured to estimate and/or record a mood of a patient, and to provide feedback on past mood history and to provide mood data to a clinician and/or the cognitive analytics engine. The mood tracker enables them to get an overview of their mood over the last several days, and also enables them to communicate their moods more easily to their care partner and clinicians. Besides a global mood, the patient is able to give feedback about feelings for more specific areas (such as work, news, family, etc.), and this information can then be sent to the clinician/caregiver so they can then follow up to understand the patient's needs better. It also includes a weekly/monthly overview so that patients can see longer term trends. This may be particularly helpful in downward trends to help catch patients early with depression, and for patients who are experiencing a temporary downturn, they will be able to see that previous downturns did not last long and may be able to rebound more quickly.

The social media module is provided to facilitate communication with family, friends and support groups. This creates a timeline, and enables communication with family and friends, as well as a way to keep up with updates from family and friends via photos and videos. This also enables access to information from support groups, including FAQs, and being able to directly make queries that can be answered by other support group members. It also allows patients to find others who have received a similar diagnosis and be able to share tips and encouragement. This can aid in reducing feelings of isolation and loneliness.

The gamification system awards points for completion of therapy or tasks, and rewards for achieving specific points goals, and a comparative score based on treatment progress with respect to other patients with similar diagnosis. Points are given for completing therapy or other tasks (like checking in consistently, or adhering to medication taking schedules). These points can be used to purchase items and upgrades in a virtual world. For example, the user could have a garden, or a farm and be able to purchase new crops or flowers. The user can also get general information about how they have scored in comparison with other users who have been given a similar diagnosis. The comparison can be selected by an algorithm for a measure where users are doing better than average so that they are encouraged to continue adhering to the therapy.

The diet tracking module is also configured to collect consumption data and provide dietary recommendations. A questionnaire can be given to the users to determine their baseline regular consumption and then using an algorithm, the system can suggest which items they need to add to their diet. Further questions can be asked to see if they are successfully adding items and if there is any correlation with items consumed and cognitive performance The system may also include some visual recognition so that users can take photos of what they are eating and the nutritional content can be computed.

To further illustrate the system an example of a clinical scenario which demonstrates the potential value of a personalized cognitive system will be described involving the case of MCI or AD (Alzheimer's disease) patients. Various cognitive behavioural therapies are recommended in various clinical and health care systems across the world. The key problem in recommending various behavioural therapies is that the clinicians and doctors don't have a platform to monitor the effect of the therapy or the improvement/responsiveness of the therapy on the patient. In this scenario, the personalized cognitive platform 50 can help guide the therapeutic decision-making which would enable the clinicians and doctors to have a report on which of the cognitive behavioural therapy is working on the patient and which therapy the patient is well responding to and accordingly titrate the behavioural therapy. On the other hand, therapeutic agents available for MCl/AD treatment, the optimal drug of choice as a first-line agent (prioritization of therapies), depends on the level and stage of AD, along with physiological response to therapy. The prescribed classes of medications for AD include Aricept, Razadyne, Namenda, Exelon, Namzaric, Aduhelm, and drugs that can reduce irregular amyloid beta and tau protein accumulation. However, most patients cannot tolerate these different agents at once. Furthermore, the titration of therapy to an optimal dose or switching to another therapeutic agent depends heavily on the behavioural response and physiological parameters. In addition, the monitoring of the patient also helps to estimate the potential therapeutic benefit of certain therapeutic agents to track the side effects of the drugs on the patient. Table 1 presents the starting dose, the optimal dose, and side effects of several classes of Alzheimer's Disease (AD) drugs.

TABLE 1
The starting dose, the optimal dose, and side effects
of several classes of Alzheimer's Disease drugs.

| | | | | MANU- |
|---|---|---|---|---|
| | DRUG | | COMMON | FACTURER'S |
| DRUG | TYPE | HOW IT | SIDE | RECOMMEND |
| NAME | AND USE | WORKS | EFFECTS | DOSAGE |
| Aricept ® | Choline- | Prevents | Nausea, | Tablet*: Initial |
| (donepezil) | sterase | the break- | vomiting, | dose of 5 mg |
| | inhibitor | down | diarrhoea, | once a day; may |
| | prescribed | of acetyl- | muscle | increase dose |
| | to treat | choline | cramps, | to 10 mg/day after |
| | symptoms | in the | fatigue, | 4-6 weeks if |
| | of mild, | brain | weight loss | well tolerated, |
| | moderate, | | | then to 23 |
| | and severe | | | mg/day after at |
| | Alzheimer's | | | least 3 months. |
| | | | | Orally |
| | | | | disintegrating |
| | | | | tablet*: |
| | | | | Same dosage as |
| | | | | above (not |
| | | | | available in 23 mg). |
| Exelon ® | Choline- | Prevents | Nausea, | Capsule*: Initial |
| (riva- | sterase | the break- | vomiting, | dose of 3 |
| stigmine) | inhibitor | down | diarrhoea, | mg/day (1.5 mg |
| | prescribed | of acetyl- | weight loss, | twice a day); |
| | to treat | choline | indigestion, | may increase |
| | symptoms | and | muscle | dose to 6 mg/day |
| | of mild to | butyryl- | weakness | (3 mg twice a |
| | moderate | choline | | day), 9 mg/day |
| | Alzheimer's | (a brain | | (4.5 mg twice a |
| | (patch is | chemical | | day), and 12 mg/ |
| | also for | similar to | | day (6 mg |
| | severe | acetyl- | | twice a day) at |
| | Alzheimer's) | choline) | | minimum 2- |
| | | in the | | week intervals |
| | | brain | | if well tolerated. |
| | | | | Patch*: Initial |
| | | | | dose of 4.6 mg |
| | | | | once a day; may |
| | | | | increase dose |
| | | | | to 9.5 mg once |
| | | | | a day and 13.3 |
| | | | | mg once a day |
| | | | | at minimum 4- |
| | | | | week intervals if |
| | | | | well tolerated. |
| Namenda ® | N-methyl | Blocks | Dizziness, | Tablet*: Initial |
| (memantine) | D-aspartate | the toxic | headache, | dose of 5 mg |
| | (NMDA) | effects | diarrhoea, | once a day; may |
| | antagonist | associated | con- | increase dose |
| | prescribed | with | stipation, | to 10 mg/day |
| | to treat | excess | confusion | (5 mg twice a |
| | symptoms | glutamate | | day), 15 mg/day |
| | of moderate | and | | (5 mg and 10 |
| | to severe | regulates | | mg as separate |
| | Alzheimer's | glutamate | | doses), and 20 |
| | | activation | | mg/day (10 mg |
| | | | | twice a day) at |
| | | | | minimum 1-week |
| | | | | intervals if |
| | | | | well tolerated. |
| | | | | Oral solution*: |
| | | | | Same dosage |
| | | | | as above. |
| | | | | Extended-release |
| | | | | capsule*: |
| | | | | Initial dose of 7 |
| | | | | mg once a day; |
| | | | | may increase |
| | | | | dose to 14 |
| | | | | mg/day, 21 mg/ |
| | | | | day, and 28 |
| | | | | mg/day at |
| | | | | minimum 1-week |
| | | | | intervals if well |
| | | | | tolerated. |
| Namzaric ® | NMDA | Blocks the | Headache, | Extended-release |
| (memantine | antagonist | toxic | nausea, | capsule*: |
| and | and | effects | vomiting, | Initial dose of 28 |
| donepezil) | choline- | associated | diarrhoea, | mg memantine/ |
| | sterase | with | dizziness, | 10 mg donepezil |
| | inhibitor | excess | anorexia | once a day if |
| | prescribed | glutamate | | stabilized on |
| | to treat | and | | memantine and |
| | symptoms | prevents | | donepezil. |
| | of moderate | the break- | | If stabilized |
| | to severe | down | | on donepezil |
| | Alzheimer's | of acetyl- | | only, initial dose |
| | | choline | | of 7 mg |
| | | in the | | memantine/10 |
| | | brain | | mg donepezil |
| | | | | once a day; may |
| | | | | increase dose |
| | | | | to 28 mg |
| | | | | memantine/10 mg |
| | | | | donepezil in 7 |
| | | | | mg increments |
| | | | | at minimum 1- |
| | | | | week intervals |
| | | | | if well tolerated. |
| | | | | Only 14 mg |
| | | | | memantine/10 mg |
| | | | | donepezil and 28 |
| | | | | mg memantine/ |
| | | | | 10 mg donepezil |
| | | | | available as |
| | | | | generic. |
| Razadyne ® | Choline- | Prevents | Nausea, | Tablet*: Initial |
| (galant- | sterase | the break- | vomiting, | dose of 8 |
| amine) | inhibitor | down | diarrhoea, | mg/day (4 mg |
| | prescribed | of acetyl- | decreased | twice a day); |
| | to treat | choline | appetite, | may increase |
| | symptoms | and | dizziness, | dose to 16 |
| | of mild to | stimulates | headache | mg/day (8 mg |
| | moderate | nicotinic | | twice a day) and |
| | Alzheimer's | receptors | | 24 mg/day (12 mg |
| | | to release | | twice a day) at |
| | | more | | minimum 4-week |
| | | acetyl- | | intervals if |
| | | choline | | well tolerated. |
| | | in the | | Extended-release |
| | | brain | | capsule*: |
| | | | | Same dosage |
| | | | | as above but |
| | | | | taken once a day. |

To further illustrate the practical use of the system consider the following workflow/clinical scenario. A 50-year old male is diagnosed with early onset AD. He has been having memory loss, some difficulty in performing instrumental activities of daily life (managing finances, social issues etc.), has sleep issues and overall BMI is higher than required. The patient is prescribed a drug named Aricept by a clinician. When the patient is on-boarded on the platform, the patient's (initial) profile is generated by capturing general, behavioural, clinical and lab/Physiological inputs by patients, caregivers and clinicians. Once the inputs are captured these are then moved to a data aggregation layer for pre-processing input data where the profile of the patient based on main four different inputs are generated which helps to form a baseline to the patient. Then the patient receives digital cognitive therapy via the digital cognitive therapy delivery module executing on the patient facing App which has 4 MOAs all together for few weeks to understand which MOA the patient responds well to. All digital cognitive biomarkers are captured for each MOA and this is sent to the cognitive analytics engine which gives the baseline of the patient, each MOA pointer, average MOA pointer, aide effects reported by the patient pointer, and a cognitive therapeutic report. All this data is then moved to the personalised cognitive digital model for the patient which has an alert generated with explanations when the effect of the therapy does not fulfil the expectation or there are severe side effects. The alert and the explanations will be sent to the cognitive therapeutic platform.

A week later after on-boarding and the beginning the medication, the patient started reporting nausea and dizziness side effects. Based on continuous real-time monitoring of patient-reported symptoms and the Side Effect Pointer, a physician using the personalized cognitive platform is alerted and proceeds to titrate the dose until an optimal drug dosage is identified for the patient (e.g. by minimising of reported side effects and change in the side effects pointer).

At the same time, digital cognitive biomarkers captured that the patient was not compliant to the scheduled physical exercises from the Wellness module. Even after the optimal drug dosage was set and no more side effects reported, the patient still continued to skip some exercises and the caregiver was notified to support and encourage the patient to complete exercises by accompanying the patient during the video-classes. Hence, the compliance to the therapy was improved.

Finally, few weeks later the cognitive analytics engine detected that the patient experienced increasing difficulties with one cognitive game while progressing well with others. Therefore, it recommended an increase in the dosage (i.e. time) of playing this game from 10 to 20 minutes per session while decreasing the difficulty coefficient determining the game speed and rule complexity. As a result, the patient showed improved game performance and the cognitive analytics engine forecasted a higher expected positive effect from MOA1 of the digital therapy.

Embodiments of the personalized cognitive platform are able to titrate 3 important features: First, the MOAs which can be a combination of MOAs or a singular MOA to which the patient is well responding and improving; Second, it can titrate the educational content based on the preferences of the patient, behavioural feedback of the patient, and other co morbid conditions (sleep issues, anxiety issues, etc.); Third, it can titrate the medication management based on the SEP which enables the clinician to intervene and change the dosage up/down titrate the dosage until it reaches a dosage optimal for the patient. Real-time, continuous, physiology based remote monitoring system could be incredibly effective in guiding clinical decision making The titration completes and then is sent to the personalised cognitive digital model for the patient taking in all the feedback and delivered to patient in the next therapy session.

In conclusion, when a patient is prescribed certain therapies, the personalized cognitive platform would take the data acquired from different resources (baseline pointers, individual and average MOA pointers, Side effect pointers, physiological parameters from sensors, medication regimen, electronic medical records, etc.) and together with the known positive and negative effects (on behavioural responses of therapies, on physiological parameters as well as patient- reported symptoms) to derive additional features including the MOAP average, MOA1P, MOA2P, MOA3P, MOA4P, SEP and CTR as described above. The system can help to guide the clinician/caregiver in therapeutic decision making and to better manage the patient on which MOA is patient well responding to and which therapy is helping the patient improve cognition and quality of life, optimal drug dosage of the patient, after the introduction of any therapies. The system even translates related educational content to the patient. The clinician will then be able to titrate the MOAs (either singular MOA or a combination of MOA), related content education for patient, Medication titration accordingly. Consequently, this will improve the prognosis of patients and ultimately be translated high outcomes to the patient and also to economic benefit.

FIG. 3 is a flow chart of use scenarios of the system according to an embodiment. This illustrates a ‘Just-in-Time Intervention’ in which real-time feedback is provided to the clinicians and caregivers allowing early interventions; thus improving adherence and compliance of the digital therapy modules. FIG. 3 shows the system 310 and a caregiver 312, patient 314 and clinician 316 and illustrates 2 scenarios. Scenario 1 is real-time monitoring of symptoms/titrating medication and scenario 2 is ensuring compliance of the therapy and physical activities.

In scenario 1, a processor 320 uses a push notification system 322 to alert the patient 314 and caregiver 312 to take medication. The patient can also report any symptoms which are provided to processor 320. In this scenario the system determines 322 if symptoms are above threshold for reporting (e.g. symptoms are worse than expected) or if an important medicine is continuously being missed/not taken. In the case of continuously missed medication a push notification service 334 is used to alert the caregiver 312, and similarly a push notification service 344 may be used to alert the clinician 316 if the symptoms are worse than expected. This real-time alerting allows the clinician to find the optimal drug dose for the patient, and when titrating is required. Finally a monitoring report of new and/or extreme symptoms is then saved into data lake 350.

Scenario 2 illustrates ensuring compliance of the therapy and physical activities 340. Data from the patient 320 is processed and an aggregated weightage score related to therapy & wearable biomarker is calculated 342. The system determines the compliance of the patient and a push notification service 344 is used to send an alerts physician. This provides immediate insights to the treating physician so that action may be taken to improve adherence and compliance to the therapy. A compliance report and score monitoring data 346 is generated and stored in a data lake 350.

Embodiments of the system may be provided as a computational system configured to provide a personalized cognitive therapeutic system, or to implement embodiments of the method for providing a personalized cognitive therapeutic system. Embodiments may be provided as a computer program product comprising computer executable instructions for providing a personalized cognitive therapeutic treatment system. The computing system may comprise a range of computing apparatus including a cloud based computing platform 70, mobile computing apparatus 80 including tablet, smart phone and laptop computing apparatus, wearable computing apparatus 88, and any associated peripheral devices. Other computing apparatus such as desktop computing apparatus, all-in-one computing apparatus, distributed computing apparatus and server based computing apparatus may also be used.

In one embodiment a local computing apparatus 80 is used by a clinician or patient which provides an interface to components of the system executing on a remote, web, or cloud based computing apparatus 70. Additional computing devices, wearables 88 or medical devices are also configured to send data to the remote, web, or cloud based computing apparatus, either directly or via the local computing apparatus. Each computing apparatus comprises at least one processor and a memory operatively connected to the processor, and the computing apparatus is configured to perform the method described herein.

A computing apparatus 70, 80, may comprise one or more processors 72, 82, one or more memories 74, 84, external storage devices, input/output devices (e.g., monitor, keyboard, disk drive, network interface, internet connection, etc.) and associated peripheral devices. The computing apparatus and computing system may also include circuitry or other specialized hardware for carrying out some or all aspects of the processes. The computer system may be a distributed system including server based systems and cloud-based computing systems. In some operational settings, the computing system may be configured as a system that includes one or more units, each of which is configured to carry out some aspects of the processes either in software, hardware, or some combination thereof. For example, the user interface of the digital cognitive therapy delivery module 30 may be provided on a mobile computing device 80 such as a smart phone, a desktop computer or tablet computer, whilst the cognitive analytics engine including training or use of AI and ML models may be performed on a cloud based server system and the user interface is configured to communicate with such servers. A clinician user interface for the personalised cognitive platform 50 may also be provided which communicates with the cloud based servers 70. User interfaces including dashboards may be provided as a web portal or an App on a smart phone, desktop or tablet computer, allowing a user on one computing apparatus to upload data which may be processed on a remote computing apparatus or system (e.g., server or cloud system) and which provides the results or report back to the user, or to other users (e.g. caregivers) on other computing apparatus.

The one or more processors 72, 82 may comprise one or more Central Processing Units (CPUs) including single CPU (core) or multiple CPU's (multiple core), Graphical Processing Units (GPUs), parallel processors, or vector processors. The one or more processors may be operatively connected to the one or more memories which store instructions to configure the processor to perform embodiments of the method. A CPU may comprise an Input/Output (I/O) Interface, an Arithmetic and Logic Unit (ALU) and a Control Unit and Program Counter element which is in communication with input and output devices through the I/O Interface. The I/O interface may be connected to input and output devices such as a display 86, a keyboard, a microphone, speaker, a camera or a mouse. The I/O Interface may comprise a network interface and/or communications module for communicating with an equivalent communications module in another device using a predefined communications protocol (e.g. Wi-Fi, Ethernet, Bluetooth, Zigbee, IEEE 802.15, IEEE 802.11, TCP/IP, UDP, etc.). For example mobile computing apparatus 80 may communicate with wearable computing apparatus 88 over a Bluetooth connection, and the mobile computing apparatus may communicate with the cloud based computing apparatus 70 over a Wi-Fi connection to a router which provides a backbone connection to the cloud based computing apparatus 70. The communications module may also be configured to communicate with medical devices such as EEG, heart rate and blood pressure monitors, as well as laboratory management systems and electronic health records to obtain physiological and laboratory based measures, test results, and medical history data (genetic, microbiome, vitals, metabolism, blood, cerebral spinal fluid, protein biomarkers, etc.).

Memory 74, 84 is operatively coupled to the processor(s) 72, 82 and may comprise RAM and ROM components, and may be provided within or external to the device or processor module. Multiple memory devices may be provided and the memory may include a disk storage device such as a solid state disk (SSD), and/or a media drive unit configured to read/write a removable computer-readable medium. The memory may be used to store the operating system, programs, software modules, instructions and/or data, including programs or data downloaded from a website or read from media inserted in the media drive unit. The processor(s) may be configured to load and executed the programs, software modules or instructions stored in the memory.

Various components of the system may use Artificial Intelligence (AI) and Machine Learning (ML) methods, for example for classifying data, predicting expected effects, and therapy adjustments. These may include Artificial Intelligence and Machine Learning methods to build a classifier (or set of classifiers), perform clustering, build regression models, etc, including using reference data sets including test and training sets, and may include deep learning methods using multiple layered classifiers and/or multiple neural networks. The AI/ML models may use features identified by various signal processing, mathematical and statistical techniques or features may be determined by the AI/ML models, and various algorithms may be used including linear classifiers, regression algorithms, support vector machines, neural networks, Bayesian networks, etc. Ensemble method may be used to combine results from multiple AI/ML models. Various software languages and ML libraries may be used to build the AI/ML models including, TensorFlow, Theano, Torch, PyTorch, Keras, Deeplearning4j, Java-ML, scikit-learn, Spark MLlib, Apache MXnet, Azure ML Studio, AML, MATLAB, etc, and the application may be written in high level languages such as Python, R, C, C++, C#, Java, Swift etc., and may use function libraries and toolkits. Data storage and data exchange may be performed using database systems, languages and protocols (e.g. Oracle, MySQL, SQL, JSON, etc.).

For building diagnostic models as part of the cognitive analytics engine 40 and MOA management within Personalized Cognitive Platform 50, different Artificial intelligence (AI) and Machine Learning (ML) algorithm or methods may be used including supervised Machine Learning algorithms using the digital cognitive biomarkers 31. These AI/ML methods include, but are not limited to, regression, support vector machine (SVM), ensemble methods like—random forest, boosting algorithms (such as AdaBoost, XGBoost, catBoost). Ensemble methods, particularly eXtreme Gradient Boosting (XGBoost) algorithm proved advantageous for tasks involving biomedical and health informatics data. An ensemble of models can also be used to improve performance In these cases multiple models may each make a prediction or classification, and a voting scheme used to select a result, for example using a majority rule. In some embodiments deep learning methods may be used in cases where models with hand crafted and hard coded features do not show expected/sufficient performance including, but not limited to, recurrent and convolutional neural networks, long short-term memory (LSTM) and others. The particular architecture and configuration of deep neural networks (i.e. the number of layers and nodes/dimensions) depend on properties of the available data. Additionally for personalization of the patient cognitive digital model, predictive models may be based on Reinforcement Learning, Generative modelling and rule-based/statistical algorithms. For MOA2, MOA3 and MOA4 models include rule-based/statistical algorithms, Natural Language Processing (NLP) methods, Bidirectional Encoder Representations from Transformers (BERT), and image processing & recommendation. These algorithms (especially the deep learning models) learn from the data and help in mapping the digital cognitive biomarkers with the target variables which assists in personalisation of the digital therapy at individual level. To train an AI/ML model, the following steps are normally performed:
Pre-processing the data, which includes data quality techniques/data cleaning to remove any label noise or bad data and preparing/normalising the data so it is ready to be utilised for AI training and validation;
Extract features if required by the AI/ML model;
Choosing the model configuration, including model type, model architecture and Machine Learning hyper-parameters;
Split the data into training dataset, validation dataset and/or test dataset;
Train the model on the training dataset—during the training process, many models are produced by adjusting and tuning the model configurations in order to optimise the performance of model according to an accuracy metric; each training iteration is referred to as an epoch, with the accuracy estimated and model updated at the end of each epoch;
Choosing the best “final” model, or an ensemble of models whose results are combined to generate a result, based on the model(s) performance on the validation dataset; the model is then applied to the “unseen” test dataset to validate the performance of the final AI model.

Cross-validation and regularization may also be performed. A range of cross-validation methods may be used to mitigate potential overfitting, reduce bias in the data and improve generalizability of the model including, but not limited to, k-fold cross-validation, leave-one-out cross-validation (LOOCV), leave-all-out time-series cross-validation and leave-one-out time-series cross-validation where appropriate and best suits the task. Different loss functions will implemented depending on the task and data properties (e.g. class probabilities) including, but not limited to, root mean square error, R-squared, binary and multiclass cross-entropy, Kullback-Leibler divergence etc. Deep neural networks may be regularized using different techniques including, but not limited to, L2 (i.e. weight decay) and L1 weigh regularization, dropout, data augmentation, etc.

In some embodiments, the AI/ML methods may use feature selection. Prior to training predictive models with Machine Learning algorithms, feature selection procedures may be executed using various methods including, but not limited to, evaluating the strength of statistical associations with Pearson's and Spearman's rank correlation coefficients between digital cognitive biomarkers 31 and various outcomes (e.g. neuropsychological test scores), one-way analysis of variance (ANOVA) test and/or Kruskal-Wallis rank sum test for categorical between-class comparisons (presence/absence of specific symptoms), etc. The relative importance of predictors may be also evaluated using multiple generalized linear regression models and Lasso regression.

The performance of models predicting numeric outcomes (e.g. neuropsychological test scores) will be evaluated using a range of metrics including, but not limited to, mean absolute error (MAE), root mean square error (RMSE), mean absolute percentage error (MAPE), and the coefficient of determination (R-squared) where appropriate and best suits the task. The performance of models predicting categorical outcomes (e.g. symptom classification) will be evaluated using a range of metrics including, but not limited to, receiver operating characteristic curve (ROC), area under the ROC curve (AUC), accuracy, sensitivity (recall), specificity, positive predictive value (precision), negative predictive value, F1-score, macro F1-score, Cohen's kappa where appropriate and best suits the task.

Once models are built and deployed, a posteriori feature importance may be evaluated and used for further model tuning, refining and personalization (e.g. for different individuals different digital cognitive biomarkers may be most indicative for future cognitive training progress). For decision tree algorithms such as Random Forest or XGBoost, the contribution of each feature to improved accuracy can be estimated. Alternatively SHapley Additive exPlanations (SHAP) can be used.

Embodiments of the system are designed to either maintain or improve cognitive functioning in patients with either Mild Cognitive Impairment (MCI), Alzheimer's Disease (AD), prodromic AD, or other neurodegenerative disorders. The system may also be used by healthy elderly to maintain or improve their cognition. The goal in particular is to improve or maintain processing speed, executive function, attentional control, perception (across all senses), sustained alertness, metacognition, immediate and delayed memory (short-term and long-term), overall cognitive functioning, and daily activity functioning. Apart from targeting cognitive health, the system use results in improved physical fitness, psychological wellbeing (mood) and quality of life (QoL) in MCI, AD and other patients with neurodegenerative disorders. The system also can improve the wellbeing and QoL of caregivers, and help clinicians provide more timely and appropriate healthcare.

As explained herein, a personalised cognitive digital therapy model is generated which generate a range of metrics from a range of digital cognitive biomarkers obtained from multiple digital therapies, along with behavioural and physiological biomarkers obtained from wearable and medical devices. A cognitive analytics engine uses Artificial Intelligence and/or Machine Learning techniques to generate a range of metrics from a range of digital cognitive biomarkers obtained from digital therapies, along with behavioural and physiological biomarkers obtained from wearable and medical devices. Model predictions and adjustments to medications and treatments are provided to a personalised cognitive platform to personalise the cognitive digital therapy model for each patient. Proposed changes may be reviewed and adjusted by clinicians. The personalised cognitive platform features a highly optimized cognitive therapeutic and monitoring solution and provides accurate prediction of deterioration for just-in-time intervention and titration.

Embodiments of the system use automated digital diagnostics to passively look at several different aspects of behaviour without giving the user a direct behavioural test. This can include but is not limited to speech vocal recordings, typing style, patterns of App usage, and physical activity and sleep. The system captures digital cognitive biomarkers from the digital cognitive therapies, such as number of memories generated in the reminiscence therapy, level achieved within each cognitive game, number of articles read in the learning module, or number of questions about the articles that have been answered correctly, as well as number of exercises, or diet recommendations completed. The system is also able to take the input from the laboratory tests, including but not limited to cerebral spinal taps, blood tests (including those for tau, amyloid beta and other neurodegenerative biomarkers), doctor's opinions, genetic analysis (such as alleles that are linked to neurodegenerative disorders like APOE variants), and gold-standard neuropsychological measures given using paper and pencil tests. Model predictions of how the metrics relate to other measures such as measures of ability to function in daily life (activities of daily living and instrumental activities of daily living), cognitive ability, depression, and behavioural impairment can be validated and updated each time a traditional gold standard pencil and paper tests are given by a clinician.

The system is iterative and uses a continuous feedback loop to personalise the personalised cognitive digital therapy model for the patient including adjustment of the digital cognitive therapies provided by the digital cognitive therapy module. As these therapies are applied, additional digital biomarker and behavioural and physiological data are collected and fed back into the cognitive analytics engine to assess treatment performance with respect to expected performance, thus allowing further evaluation and adjustment of the personalised cognitive digital therapy model. Digital cognitive biomarkers captured by the digital cognitive therapy delivery module are used for automated or semi-automated personalization of the digital therapy modules (MOAs) to personalise the treatment of the patient. The primarily focus is on MOA management instead of medication and educational content management because adjustment, titration and personalization of digital therapies can be automated and requires less human involvement.

A patient's performance scores and measures are linked to cognitive treatment targets (cognitive demands—e.g. processing speed, or response to particular types of problems). An ensemble of AI and

ML models learn from gameplay data of how a player's cognitive performance changes depending on variable game parameters including game speed, obstacle types, level rules, instruction complexity, and other game aspects which constitute multitude of variations within the same level. As a result, AI and ML models can predict what effect each variation of game parameters have on performance and accordingly adjust game parameters to maximize the anticipated positive effect on training cognitive abilities from the digital cognitive therapies. This can be both by decreasing or increasing difficulty. The cognitive analytics engine learns continuously and the process of digital cognitive therapy adjustment is iterative, and hence the user is continuously kept at a level of difficulty that is optimal for learning.

For example, cognitive analytics engine may use a model that determines what aspects of the game are leading to the user scoring lower points, or progressing more slowly towards the goal point of the game. This model can then be used to adjust the obstacles that are causing difficulty for the player. e.g. if a user is having trouble doing complex visual search, the items to search for can be adjusted to be more distinct from the items to ignore, or if the user is reacting too slowly to avoid a certain obstacle, the speed can be individually adjusted for that obstacle.

Beyond direct digital cognitive biomarkers of cognitive functioning (e.g. reaction time, response to increasingly difficult problems), the system is configured to evaluate and predict a patient's cognitive scores on gold standard neuropsychological tests (e.g. MMSE, Clinical Dementia Rating). These predictions can help clinicians determine when intervention is needed either because the patient is declining faster than expected or because the treatment is working better than expected so that adjustments need to be made. All types of cognitive, physiological and behavioural digital cognitive biomarkers captured by the system (game performance, types of errors, reaction time, HRV metrics, skin conductance response metrics and others) can be used to predict these neuropsychological test scores. For predicting neuropsychological test scores the system can use population-based and personalized ML models, where the former model is trained using data on standardized neuropsychological tests from other people in the same (or closest) population group, while the last model is trained using past scores from the same person. The system can determine which of these features is most predictive for a particular patient and weight these features more highly when making these predictions.

Embodiments of the personalised cognitive therapeutic system described herein thus monitors the effect of therapy on individual patient and can provide valuable insights to the caregiver/clinician for better therapeutic decision making Through the use of a range of digital therapies using different MOA, a range of digital cognitive biomarkers can be collected and analysed. Patient state and capabilities, along with the side effects the patient is suffering can be identified so that targeted digital treatments can be selected, for example to improve cognitive function, mood, and/or physical function, and thus improve the patient's quality of life. In some embodiments the provision of targeted non-pharmacological interventions may allow the dose of the pharmacological interventions to be reduced, thus reducing side effects which may contribute to an improved quality of the life for the patient.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, middleware, platforms, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The steps of a method or algorithm described in connection with the embodiments disclosed herein may be embodied directly in hardware, in a software module executed by a processor, or in a combination of the two, including cloud based systems. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, or other electronic units designed to perform the functions described herein, or a combination thereof. Various middleware and computing platforms may be used.

Software modules, also known as computer programs, computer codes, or instructions, may contain a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media. In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Further, it should be appreciated that modules and/or other appropriate means for performing the methods and techniques described herein can be downloaded and/or otherwise obtained by computing device. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a computing device can obtain the various methods upon coupling or providing the storage means to the device. Moreover, any other suitable technique for providing the methods and techniques described herein to a device can be utilized.

The reference to any prior art in this specification is not, and should not be taken as, an acknowledgement or any form of suggestion that such prior art forms part of the common general knowledge.

It will be understood that the terms “comprise” and “include” and any of their derivatives (e.g. comprises, comprising, includes, including) as used in this specification, and the claims that follow, is to be taken to be inclusive of features to which the term refers, and is not meant to exclude the presence of any additional features unless otherwise stated or implied.

In some cases, a single embodiment may, for succinctness and/or to assist in understanding the scope of the disclosure, combine multiple features. It is to be understood that in such a case, these multiple features may be provided separately (in separate embodiments), or in any other suitable combination. Alternatively, where separate features are described in separate embodiments, these separate features may be combined into a single embodiment unless otherwise stated or implied. This also applies to the claims which can be recombined in any combination. That is a claim may be amended to include a feature defined in any other claim. Further a phrase referring to “at least one of” a list of items refers to any combination of those items, including single members. As an example, “at least one of: a, b, or c” is intended to cover: a, b, c, a-b, a-c, b-c, and a-b-c.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A computational personalized cognitive therapeutic system comprising one or more processors and one or more associated memories configured to implement:
a patient clinical database comprising a data acquisition interface configured to receive and store input data for a plurality of patients from a plurality of heterogeneous data sources, the input data for a patient comprising: personal particular and sociodemographic data;
patient medical history and background health data; medication data; clinical data; and wearable data comprising behavioural data and physiological data, wherein the input data sources for a patient comprise one or more wearable devices and one or more of an electronic medical record, a digitised caregiver record, a laboratory information management system, a clinical database, and/or a patient on-boarding module;
a data aggregation and pre-processing module configured to pre-process the input data in the patient clinical database and generate a patient profile for each patient;
a digital cognitive therapy delivery module configured to deliver a plurality of therapies according to a personalised cognitive digital therapy model to a patient using one or more computing devices, each therapy having a different mechanism of action (MOA) and comprising a plurality of adjustable parameters, and the digital cognitive therapy delivery module is further configured to collect a plurality of digital cognitive biomarkers for each therapy, a plurality of behavioural and physiological biomarkers from one or more wearable devices and/or the one or more computing devices, and to measure interactions of the patient with the digital cognitive therapy delivery module, wherein the plurality of therapies comprises at least a cognitive stimulation game therapy, a guided learning therapy, a reminiscence therapy and a physical and mental wellness therapy;
a cognitive analytics engine configured to process the patient profile, digital cognitive biomarkers, and the behavioural and physiological biomarkers using an ensemble of population-based and personalised prediction models trained using a plurality of Artificial Intelligence (AI) and Machine Learning (ML) methods, and which is configured to generate a plurality of metrics to characterize a current cognitive state of the patient and estimate the potential future improvement comprising the probability and size of an expected effect, wherein the plurality of metrics are generated on demand or at least once per day; and
a personalised cognitive platform configured to use the plurality of metrics to personalize the personalised cognitive digital therapy model for each patient by adjusting one or more of the plurality of parameters for one or more of the plurality of therapies to maximise the estimated effect level, and to use the metrics to generate one or more alerts if a therapy does not meet an expected threshold effect level or a side effect exceeds a threshold side effect level to enable adjustment of a medication by a clinician,
wherein the system iteratively refines the personalised cognitive digital therapy model for each patient over time by selecting specific therapies from the plurality of therapies and adjusting the associated adjustable parameters, and obtaining an estimate effect of the adjustments, and after delivery of an adjusted treatment by the digital cognitive therapy delivery module, the cognitive analytics engine generates the plurality of metrics to assess actual effects compared to estimated effects in order to further refine the personalised cognitive digital therapy model by the personalised cognitive platform.

2. The system as claimed in claim 1, wherein the data aggregation and pre-processing module is configured to perform data cleaning, dimensionality reduction and data transformation to prepare the input data for further analysis and use by the cognitive analytics engine.

3. The system as claimed in claim 1, wherein:
the plurality of digital cognitive biomarkers for the cognitive stimulation game therapy comprises one or more of a game specific performance, finger tapping and finger movement related biomarkers, reaction time between a stimulus exposure and a response, and a proxy index of a cognitive load;
the plurality of digital cognitive biomarkers for the guided learning therapy comprises one or more of a quiz result, an answer confidence, a speed of information processing of content, or a time spent with content;
the plurality of digital cognitive biomarkers for the reminiscence therapy comprises one or more of a language marker derived from textual analysis or audio analysis, a speech characteristic derived from audio data of the patient; and
the plurality of digital cognitive biomarkers for the physical and mental wellness therapy comprises one or more of an access frequency of content, time spent with content, an application opening and closing frequency, a task completion within an allotted time, a compliance with an allotted task, a direct feedback from the patient on the likeability and difficulty of the therapy via a questionnaire, and an emotional expression capture indicating a level of enjoyment.

4. The system as claimed in claim 1, wherein the plurality of behavioural and physiological biomarkers comprise one or more of an movement biomarker obtained from an accelerometer and/or a gyroscope, an electrodermal activity or skin conductance biomarker, a photoplethysmography or blood volume pulse biomarker, a heart rate biomarker, a heart rate variability biomarker, a skin temperature biomarker, a facial expression biomarker, an eye tracking biomarker, and a neural activity biomarker.

5. The system as claimed in claim 1, wherein the plurality of metrics comprise:
a cognitive baseline pointer which is an estimate of a change in the cognitive state of the patient with respect to a baseline generated using the patient profile and an expected behaviour effect on the patient generated by the cognitive analytics engine;
a mechanism of action pointer for each of the plurality of mechanisms of action which estimates an effect level with respect to an expected effect level for the associated therapy generated by the cognitive analytics engine;
an average mechanism of action pointer which estimates an average effect of the plurality of therapies with respect to an estimate effect generated by the cognitive analytics engine; and
a side effects pointer which measures a severity of one or more side effects.

6. The system as claimed in claim 5, wherein the personalised cognitive platform comprises a MOA management module, an educational content management module and a medication/dose management module, wherein the MOA management module uses at least the mechanism of action pointers and the average mechanism of action pointer to adjust one or more of the plurality of parameters for one or more of the plurality of therapies and to adjust the dosage of each of the plurality of therapies to maximise the estimated effect level of a therapy, and wherein the content education module is configured to adjust a digital content provided to a patient based on the patient's interaction behaviour measured by the digital cognitive therapy delivery module, and the medication/dose management module is configured to record clinical data including medication and dosages and to generate suggested changes to medication and dosages using at least the side effects pointer and the cognitive baseline pointer.

7. The system as claimed in claim 6, wherein the MOA management module is configured to adjust the cognitive stimulation game therapy by adjusting one or more game parameters, game dosage and game timing, and is configured to adjust the guided learning therapy by adjusting the learn amount and timing, and learning content, and is configured to adjust the reminiscence therapy by adjusting the timing of content, content topics and stimulus and is configured to adjust the physical and mental wellness therapy by adjusting a modality, an intensity and a duration of physical exercise or mental exercise.

8. The system as claimed in claim 1, wherein the digital cognitive therapy delivery module provides a user interface on a computing device comprising:
a reminder and calendar module configured to allow patients to record reminders and to notify the patient of a scheduled therapy, and monitors therapy compliance;
a note module configured to track goals and record electronic information to assist with daily living activities;
a medication schedule module configured to record a medication schedule and track compliance;
an electronic gratitude journal;
a mood tracker configured to estimate and/or record a mood of a patient, and to provide feedback on past mood history and to provide mood data to a clinician and/or the cognitive analytics engine;
a social media module to facilitate communication with family, friends and support groups;
a gamification system which awards points for completion of therapy or tasks, and rewards for achieving specific points goals, and a comparative score based on treatment progress with respect to other patients with similar diagnosis;
a diet tracking module configured to collect consumption data and provide dietary recommendations; and
a therapy module configured to provide the plurality of therapies to the patient.

9. The system as claimed in claim 1, wherein the system comprises a cloud computing platform and the digital cognitive therapy delivery module is configured to execute on one or more patient mobile computing devices.

10. A method for providing a personalized cognitive therapeutic system comprising the steps of:
receiving and storing input data for a plurality of patients from a plurality of heterogeneous data sources in a patient clinical database using a data acquisition interface, the input data for a patient comprising: personal particular and sociodemographic data; patient medical history and background health data; medication data; clinical data; and wearable data comprising behavioural data and physiological data, wherein the input data sources for a patient comprise one or more wearable devices and one or more of an electronic medical record, a digitised caregiver record, a laboratory information management system, a clinical database, and/or a patient on-boarding module;
aggregating and pre-processing the input data in the patient clinical database and generating a patient profile for each patient;
delivering a plurality of therapies according to a personalised cognitive digital therapy model to a patient using one or more computing devices executing a digital cognitive therapy delivery module wherein each therapy has a different mechanism of action (MOA) and comprises a plurality of adjustable parameters, and the digital cognitive therapy delivery module is further configured to collect a plurality of digital cognitive biomarkers for each therapy, a plurality of behavioural and physiological biomarkers from one or more wearable devices and/or the one or more computing devices, and to measure interactions of the patient with the digital cognitive therapy delivery module, wherein the plurality of therapies comprises at least a cognitive stimulation game therapy, a guided learning therapy, a reminiscence therapy and a physical and mental wellness therapy;
processing, using a cognitive analytics engine, the patient profile, the plurality of digital cognitive biomarkers, and the plurality of behavioural and physiological biomarkers using an ensemble of population-based and personalised prediction models trained using a plurality of Artificial Intelligence (AI) and Machine Learning (ML) methods, and which is configured to generate a plurality of metrics to characterize a current cognitive state of the patient and estimate the potential future improvement comprising the probability and size of an expected effect, wherein the plurality of metrics are generated on demand or at least once per day; and
personalizing, by a personalised cognitive platform configured to use the plurality of metrics, the personalised cognitive digital therapy model for each patient by adjusting one or more of the plurality of parameters for one or more of the plurality of therapies to maximise the estimated effect level, and to use the metrics to generate one or more alerts if a therapy does not meet an expected threshold effect level or a side effect exceeds a threshold side effect level to enable adjustment of a medication by a clinician,
wherein the system iteratively refines the personalised cognitive digital therapy model for each patient over time by selecting specific therapies from the plurality of therapies and adjusting the associated adjustable parameters, and obtaining an estimate effect of the adjustments, and after delivery of an adjusted treatment by the digital cognitive therapy delivery module, the cognitive analytics engine generates the plurality of metrics to assess actual effects compared to estimated effects in order to further refine the personalised cognitive digital therapy model by the personalised cognitive platform.

11. The method as claimed in claim 10, wherein aggregating and pre-processing comprises performing data cleaning, dimensionality reduction and data transformation to prepare the input data for further analysis and use by the cognitive analytics engine.

12. The method as claimed in claim 10, wherein:
the plurality of digital cognitive biomarkers for the cognitive stimulation game therapy comprises one or more of a game specific performance, finger tapping and finger movement related biomarkers, reaction time between a stimulus exposure and a response and a proxy index of a cognitive load;
the plurality of digital cognitive biomarkers for the guided learning therapy comprises one or more of a quiz result, an answer confidence, a speed of information processing of content, or a time spent with content;
the plurality of digital cognitive biomarkers for the reminiscence therapy comprises one or more of a language marker derived from textual analysis or audio analysis, a speech characteristic derived from audio data of the patient; and
the plurality of digital cognitive biomarkers for the physical and mental wellness therapy comprises one or more of an access frequency of content, a time spent with content, an application opening and closing frequency, a task completion within an allotted time, a compliance with an allotted task, a direct feedback from the patient on the likeability and difficulty of the therapy via a questionnaire, and an emotional expression capture indicating a level of enjoyment.

13. The method as claimed in claims 10, wherein the plurality of behavioural and physiological biomarkers comprise one or more of an movement biomarker obtained from an accelerometer and/or a gyroscope, an electrodermal activity or skin conductance biomarker, a photoplethysmography or blood volume pulse biomarker, a heart rate biomarker, a heart rate variability biomarker, a skin temperature biomarker, a facial expression biomarker, an eye tracking biomarker, and a neural activity biomarker.

14. The method as claimed in claim 10, wherein the plurality of metrics comprise:
a cognitive baseline pointer which is an estimate of a change in the cognitive state of the patient with respect to a baseline generated using the patient profile and an expected behaviour effect on the patient generated by the cognitive analytics engine;
a mechanism of action pointer for each of the plurality of mechanisms of action which estimates an effect level with respect to an expected effect level for the associated therapy generated by the cognitive analytics engine;
an average mechanism of action pointer which estimates an average effect of the plurality of therapies with respect to an estimate effect generated by the cognitive analytics engine; and
a side effects pointer which measures a severity of one or more side effects.

15. The method as claimed in claim 14, wherein the personalised cognitive platform comprises a MOA management module, an educational content management module and a medication/dose management module, wherein the MOA management module uses at least the mechanism of action pointers and the average mechanism of action pointer to adjust one or more of the plurality of parameters for one or more of the plurality of therapies and to adjust the dosage of each of the plurality of therapies to maximise the estimated effect level of a therapy, and wherein the content education module is configured to adjust a digital content provided to a patient based on the patient's interaction behaviour measured by the digital cognitive therapy delivery module, and the medication/dose management module is configured to record clinical data including medication and dosages and to generate suggested changes to medication and dosages using at least the side effects pointer and the cognitive baseline pointer.

16. The method as claimed in claim 15, wherein the MOA management module is configured to adjust the cognitive stimulation game therapy by adjusting one or more game parameters, game dosage and game timing, and is configured to adjust the guided learning therapy by adjusting the learn amount and timing, and learning content, and is configured to adjust the reminiscence therapy by adjusting the timing of content, content topics and stimulus and is configured to adjust the physical and mental wellness therapy by adjusting a modality, an intensity and a duration of physical exercise or mental exercise.

17. The method as claimed in 10, wherein the digital cognitive therapy delivery module provides a user interface on a computing device comprising:
a reminder and calendar module configured to allow patients to record reminders and to notify the patient of a scheduled therapy, and monitors therapy compliance;
a note module configured to track goals and record electronic information to assist with daily living activities;
a medication schedule module configured to record a medication schedule and track compliance;
an electronic gratitude journal;
a mood tracker configured to estimate and/or record a mood of a patient, and to provide feedback on past mood history and to provide mood data to a clinician and/or the cognitive analytics engine;
a social media module to facilitate communication with family, friends and support groups;
a gamification system which awards points for completion of therapy or tasks, and rewards for achieving specific points goals, and a comparative score based on treatment progress with respect to other patients with similar diagnosis;
a diet tracking module configured to collect consumption data and provide dietary recommendations; and
a therapy module configured to provide the plurality of therapies to the patient.

18. The method as claimed in claim 10, wherein the method is implemented using a cloud computing platform and the digital cognitive therapy delivery module is configured to execute on one or more patient mobile computing devices.

19. A computer readable medium comprising instructions for causing a processor to implement the method of claim 10.
